(19) United States
(12) Patent Application Publication
(10) Pub. No.: US 2024/0425614 A1
(43) Pub. Date: Dec. 26, 2024

(54) ANTI-ADAMTS13 ANTIBODY AND USE THEREOF

(71) Applicants: Nara Medical University, Kashihara (JP); National Institutes of Biomedical Innovation, Health and Nutrition, Ibaraki City (JP); Alfresa Pharma Corporation, Osaka (JP)

(72) Inventors: Masanori Matsumoto, Kashihara (JP); Masaki Hayakawa, Kashihara (JP); Teruhito Yasui, Ibaraki City (JP); Takeharu Minamitani, Ibaraki City (JP)

(21) Appl. No.: 18/548,856

(22) PCT Filed: Mar. 3, 2022
(86) PCT No.: PCT/JP2022/009106

§ 371 Date: Sep. 5, 2023

(30) Foreign Application Priority Data
Mar. 5, 2021 (JP) 2021-035392

(51) Int. Cl. C07K 16/40; G01N 33/573

(52) U.S. Cl. CPC C07K 16/40; G01N 33/573; C07K 2317/24; C07K 2317/76; C07K 2317/92; G01N 2333/96486

(57) ABSTRACT

A preparation of an antibody or an antibody derivative that can prevent or treat acquired von Willebrand syndrome (AVWS) accompanied by a disease in need of mechanical assisted circulation. The antibody or an antibody derivative has specific binding activity against ADAMTS13, von Willebrand factor (VWF) cleaving protease, which is a causative factor of the acquired von Willebrand syndrome (AVWS) in humans, and can reduce the excessive cleavage of VWF by ADAMTS13.

This application is the U.S. National Phase under 35 U.S.C. § 371 of International Application PCT/JP2022/009106, filed Mar. 3, 2022, designating the U.S., and published in Japanese as WO 2022/186331 on Sep. 9, 2022, which claims priority to Japanese Patent Application No. 2021-035392, filed on Mar. 5, 2021, the entire content of which is incorporated herein by reference.

### SEQUENCE LISTING STATEMENT

The present application contains a Sequence Listing, which is being submitted via Patent Center on even date herewith. The Sequence Listing is submitted in a file entitled “2024-02-14_Sequence_Listing_UIPS004_001APC.txt” which was created on Feb. 14, 2024, and is approximately 57, 344 bytes in size. This Sequence Listing is hereby incorporated by reference.

### TECHNICAL FIELD

The present invention relates to an antibody having an inhibitory function against the activity of ADAMTS13, which is von Willebrand factor (VWF) cleaving protease, having activity of cleaving VWF, and pharmaceutical use of such an antibody.

### BACKGROUND ART

Von Willebrand factor (VWF) is a high-molecular plasma glycoprotein having effects of initial platelet adhesion, platelet aggregation, and coagulation factor VIII stabilization at a vascular injury site. VWF is produced by vascular endothelial cells or megakaryocytes, secreted as a multimer, and cleaved into a size appropriate for a hemostatic function by ADAMTS13, VWF cleaving protease. However, the excessive cleavage of VWF by ADAMTS13 develops von Willebrand disease (VWD) which is a congenital coagulopathy causing bleeding.

The inventors of the present invention have prepared a monoclonal antibody having specific affinity for ADAMTS13 for the purpose of treating VMD (Patent Literature 1). The inventors have showed that the obtained antibody was used to exhibit localization of human ADAMTS13 in satellite cells of the liver (Non Patent Literature 1).

In recent years, the occurrence of bleeding complications after implantation of left ventricular assist device (LVAD) has become a large problem. An important factor thereof is a loss of a high-molecular-weight multimer of VWF in patients after mechanical assisted circulation (Non Patent Literature 2). In short, unlike VMD, the congenital genetic disease, this disorder is acquired von Willebrand syndrome (AVWS) which is characterized by having bleeding diathesis neither in a previous medical history nor in a family history. In the present situation, AVWS is gaining attention with increase in the number of diseases in need of mechanical assisted circulation.

However, a method for preventing or treating AVWS has not yet been established. Therefore it is an urgent issue to establish such method.

### CITATION LIST

### Patent Literature

Patent Literature 1: Japanese Patent No. 4533995

### Non Patent Literature

Non Patent Literature 1: Uemura M, et al., Blood, 106, 922-924, 2005
Non Patent Literature 2: Meyer AL, et al., JACC Heart Fail 2141-145, 2014

### SUMMARY OF INVENTION

### Technical Problem

An object of the present invention is to provide a preparation of an antibody or an antibody derivative that can prevent or treat AVWS accompanied by a disease in need of mechanical assisted circulation.

### Solution to Problem

The present invention has achieved the object by providing an antibody or an antibody derivative that has specific binding activity against ADAMTS13, VWF cleaving protease which is considered as a causative factor of the human AVWS, and can reduce the excessive cleavage of VWF by ADAMTS13.

More specifically, the application of this case provides the following aspects in order to attain the object mentioned above: [1] an antibody or an antibody derivative thereof having a binding property to ADAMTS13 and having an inhibitory effect on von Willebrand factor (VWF) cleaving activity, comprising any heavy chain and light chain complementarity determining regions selected from the group consisting of
(1) heavy chain complementarity determining regions CDR1 (GYSFTGYT, SEQ ID NO: 1), CDR2 (INPYNGGT, SEQ ID NO: 2), and CDR3 (ARTSGYLFAY, SEQ ID NO: 3), and
light chain complementarity determining regions CDR1 (EDIYNR, SEQ ID NO: 4), CDR2 (GAT, SEQ ID NO: 5), and CDR3 (QQYWSSPLT, SEQ ID NO: 6);
(2) heavy chain complementarity determining regions CDR1 (GFSLPRYG, SEQ ID NO: 7), CDR2 (IWAGGST, SEQ ID NO: 8), and CDR3 (ARAGGSOPFDY, SEQ ID NO: 9), and
light chain complementarity determining regions CDR1 (RDINTY, SEQ ID NO: 10), CDR2 (RAN, SEQ ID NO: 11), and CDR3 (LOYDEFPWT, SEQ ID NO: 12);
(3) heavy chain complementarity determining regions CDR1 (GFSLTRYG, SEQ ID NO: 13), CDR2 (IWAGGST, SEQ ID NO: 14), and CDR3 (ARAGGSSSFDY, SEQ ID NO: 15), and
light chain complementarity determining regions CDR1 (QDINTY, SEQ ID NO: 16), CDR2 (RAN, SEQ ID NO: 17), and CDR3 (LOYDEFPWT, SEQ ID NO: 18); and
(4) heavy chain complementarity determining regions CDR1 (GFSLTGYG, SEQ ID NO: 19), CDR2 (IWADGTT, SEQ ID NO: 20), and CDR3 (ARAGGSQPFDY, SEQ ID NO: 21), and
light chain complementarity determining regions CDR1 (QDINSY, SEQ ID NO: 22), CDR2 (RAN, SEQ ID NO: 23), and CDR3 (LOYDEFPWT, SEQ ID NO: 24);
[2] the antibody or the antibody derivative according to [1], wherein the antibody or the antibody derivative is of recombinant type;
[3] the antibody or the antibody derivative according to [1] or [2], wherein the antibody derivative is selected from an engineered antibody selected from the group consisting of a humanized antibody, a chimeric antibody, a single-chain antibody, a multivalent antibody, and a multispecific antibody, or a functional fragment thereof;
[4] the antibody or the antibody derivative according to any of [1] to [3], wherein an amino acid sequence of a heavy chain variable region VH domain of the antibody or the antibody derivative is selected from the group consisting of
(1-1) the amino acid sequence of SEQ ID NO: 25, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 25,
(1-2) the amino acid sequence of SEQ ID NO: 27, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 27,
(1-3) the amino acid sequence of SEQ ID NO: 29, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 29,
(2) the amino acid sequence of SEQ ID NO: 31, or an amino acid sequence having the substitution (e.g., conservative substitution), insertion, or deletion of one or several amino acids at a moiety except for CDR1 (SEQ ID NO: 7), CDR2 (SEQ ID NO: 8), and CDR3 (SEQ ID NO: 9) in the amino acid sequence of SEQ ID NO: 31,
(3) the amino acid sequence of SEQ ID NO: 33, or an amino acid sequence having the substitution (e.g., conservative substitution), insertion, or deletion of one or several amino acids at a moiety except for CDR1 (SEQ ID NO: 13), CDR2 (SEQ ID NO: 14), and CDR3 (SEQ ID NO: 15) in the amino acid sequence of SEQ ID NO: 33, and
(4) the amino acid sequence of SEQ ID NO: 35, or an amino acid sequence having the substitution (e.g., conservative substitution), insertion, or deletion of one or several amino acids at a moiety except for CDR1 (SEQ ID NO: 19), CDR2 (SEQ ID NO: 20), and CDR3 (SEQ ID NO: 21) in the amino acid sequence of SEQ ID NO: 35;
[5] the antibody or the antibody derivative according to any of [1] to [4], wherein an amino acid sequence of a light chain variable region VL domain of the antibody or the antibody derivative is selected from the group consisting of
(1-1) the amino acid sequence of SEQ ID NO: 26, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 26,
(1-2) the amino acid sequence of SEQ ID NO: 28, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 28,
(1-3) the amino acid sequence of SEQ ID NO: 30, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 30,
(2) the amino acid sequence of SEQ ID NO: 32, or an amino acid sequence having the substitution (e.g., conservative substitution), insertion, or deletion of one or several amino acids at a moiety except for CDR1 (SEQ ID NO: 10), CDR2 (SEQ ID NO: 11), and CDR3 (SEQ ID NO: 12) in the amino acid sequence of SEQ ID NO: 32,
(3) the amino acid sequence of SEQ ID NO: 34, or an amino acid sequence having the substitution (e.g., conservative substitution), insertion, or deletion of one or several amino acids at a moiety except for CDR1 (SEQ ID NO: 16), CDR2 (SEQ ID NO: 17), and CDR3 (SEQ ID NO: 18) in the amino acid sequence of SEQ ID NO: 34, and
(4) the amino acid sequence of SEQ ID NO: 36, or an amino acid sequence having the substitution (e.g., conservative substitution), insertion, or deletion of one or several amino acids at a moiety except for CDR1 (SEQ ID NO: 22), CDR2 (SEQ ID NO: 23), and CDR3 (SEQ ID NO: 24) in the amino acid sequence of SEQ ID NO: 36;
[6] the antibody or the antibody derivative according to any of [1] to [5], wherein the antibody or the antibody derivative is selected from the group consisting of
(1-1) an antibody or an antibody derivative comprising a heavy chain (the amino acid sequence of SEQ ID NO: 37, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 37) and
a light chain (the amino acid sequence of SEQ ID NO: 38, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 38);
(1-2) a heavy chain (the amino acid sequence of SEQ ID NO: 39, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 39) and
a light chain (the amino acid sequence of SEQ ID NO: 40, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 40);
(1-3) a heavy chain (the amino acid sequence of SEQ ID NO: 41, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 41) and
a light chain (the amino acid sequence of SEQ ID NO: 42, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 42);
(2) an antibody or an antibody derivative comprising
a heavy chain (the amino acid sequence of SEQ ID NO: 43, or an amino acid sequence having the substitution (e.g., conservative substitution), insertion, or deletion of one or several amino acids at a moiety except for CDR1 (SEQ ID NO: 7), CDR2 (SEQ ID NO: 8), and CDR3 (SEQ ID NO: 9) in the amino acid sequence of SEQ ID NO: 43) and
a light chain (the amino acid sequence of SEQ ID NO: 44, or an amino acid sequence having the substitution (e.g., conservative substitution), insertion, or deletion of one or several amino acids at a moiety except for CDR1 (SEQ ID NO: 10), CDR2 (SEQ ID NO: 11), and CDR3 (SEQ ID NO: 12) in the amino acid sequence of SEQ ID NO: 44);
(3) an antibody or an antibody derivative comprising a heavy chain (the amino acid sequence of SEQ ID NO: 45, or an amino acid sequence having the substitution (e.g., conservative substitution), insertion, or deletion of one or several amino acids at a moiety except for CDR1 (SEQ ID NO: 13), CDR2 (SEQ ID NO: 14), and CDR3 (SEQ ID NO: 15) in the amino acid sequence of SEQ ID NO: 45) and
a light chain (the amino acid sequence of SEQ ID NO: 46, or an amino acid sequence having the substitution (e.g., conservative substitution), insertion, or deletion of one or several amino acids at a moiety except for CDR1 (SEQ ID NO: 16), CDR2 (SEQ ID NO: 17), and CDR3 (SEQ ID NO: 18) in the amino acid sequence of SEQ ID NO: 46); and
(4) an antibody or an antibody derivative comprising a heavy chain (the amino acid sequence of SEQ ID NO: 47, or an amino acid sequence having the substitution (e.g., conservative substitution), insertion, or deletion of one or several amino acids at a moiety except for CDR1 (SEQ ID NO: 19), CDR2 (SEQ ID NO: 20), and CDR3 (SEQ ID NO: 21) in the amino acid sequence of SEQ ID NO: 47) and
a light chain (the amino acid sequence of SEQ ID NO: 48, or an amino acid sequence having the substitution (e.g., conservative substitution), insertion, or deletion of one or several amino acids at a moiety except for CDR1 (SEQ ID NO: 22), CDR2 (SEQ ID NO: 23), and CDR3 (SEQ ID NO: 24) in the amino acid sequence of SEQ ID NO: 48);
[7] a pharmaceutical composition for preventing or treating bleeding caused by the excessive cleavage of VWF, comprising the antibody or the antibody derivative according to any one of claims 1 to 6;
[8] the pharmaceutical composition according to [7], wherein the bleeding caused by the excessive cleavage of VWF is bleeding associated with mechanical assisted circulation;
[9] the pharmaceutical composition according to [7] or [8], wherein the pharmaceutical composition comprises plural types of antibodies or antibody derivatives according to any of [1] to [6];
[10] the pharmaceutical composition according to any of [7] to [9], wherein the pharmaceutical composition inhibits the cleaving activity of ADAMTS13 against VWF;
[11] the pharmaceutical composition according to any of [7] to [10], wherein the bleeding associated with mechanical assisted circulation is acquired von Willebrand syndrome (AVWS);
[12] the pharmaceutical composition according to any of [7] to [11], wherein the mechanical assisted circulation is selected from the group consisting of extracorporeal membrane oxygenation (ECMO), implantable left ventricular assist device (LVAD), and percutaneous cardiopulmonary support (PCPS);
[13] a method for detecting and/or measuring the presence or an amount of ADAMTS13 in a biological sample, comprising the steps of:
contacting the biological sample collected from a test subject with the antibody or the antibody derivative according to any of [1] to [6] in vitro; and
detecting and/or measuring ADAMTS13 in the sample bound with the antibody or the antibody derivative;
a kit for detecting and/or measuring the presence or an amount of ADAMTS13 in the body of a test subject, comprising the antibody or the antibody derivative according to any of [1] to [6];

[15] a method for detecting and/or measuring the VWF cleaving activity of ADAMTS13 in a biological sample, comprising the steps of:
adding varying concentrations of the antibody or the antibody derivative according to any of [1] to [6] to the biological sample collected from a test subject, followed by incubation; and
detecting and/or measuring an intact VWF multimer, an intact VWF monomer, or a cleaved VWF fragment in the sample; and
[16] a kit for detecting and/or measuring an intact VWF multimer, an intact VWF monomer, or a cleaved VWF fragment in the body of a test subject, comprising the antibody or the antibody derivative according to any of [1] to [6].

### Advantageous Effects of Invention

The present invention can provide an antibody or an antibody derivative that has specific binding activity against ADAMTS13, von Willebrand factor (VWF) cleaving protease which is considered as a causative factor of the acquired von Willebrand syndrome (AVWS) in humans, and can reduce the excessive cleavage of VWF by ADAMTS13. Since the excessive cleavage of VWF by ADAMTS13 is considered as pathogenesis of the acquired von Willebrand syndrome (AVWS) in humans, the present invention can also provide a pharmaceutical composition for preventing or treating human AVWS, comprising the antibody or the antibody derivative of the present invention.

### BRIEF DESCRIPTION OF DRAWINGS

FIG. 1 is a figure showing the domain structure of ADAMTS13 which is a target substance of the antibody or the antibody derivative of the present invention. The numerals in the drawing represent amino acid numbers.

FIG. 2 is a figure showing the pathogenesis of AVWS.

FIG. 3 is a figure showing the ADAMTS13 binding activity of anti-ADAMTS13 antibodies (mouse A10 antibody and human chimeric A10c antibody).

FIG. 4 is a figure showing an inhibitory effect of an anti-ADAMTS13 antibody on the ADAMTS13 activity (A10c antibody).

FIG. 5 is a figure showing concentration-binding correlation for the ADAMTS13 binding activity of anti-ADAMTS13 antibodies (A10h/8A7 antibody and A10h/16E8 antibody).

FIG. 6 is a figure showing the ADAMTS13 activity inhibitory effect of anti-ADAMTS13 antibodies (A10h/8A7 antibody and A10h/16E8 antibody).

FIG. 7 is a figure showing change in high molecular weight VWF multimer deficiency by shear stress (FIG. 7A) and the inhibition of high molecular weight VWF multimer deficiency by the addition of A10 antibody (FIG. 7B).

### DESCRIPTION OF EMBODIMENTS

Terms used in the present invention are defined as follows:

(a) Von Willebrand Factor (VWF)

VWF is a high-molecular plasma glycoprotein having effects of initial platelet adhesion at a vascular injury site, platelet aggregation, and coagulation factor VIII stabilization. VWF is produced by vascular endothelial cells or megakaryocytes, secreted as a multimer, and cleaved into a size appropriate for a hemostatic function by ADAMTS13, a VWF cleaving enzyme.

(b) von Willebrand disease (VWD) is a congenital coagulopathy leading to bleeding tendency due to quantitative and qualitative abnormalities of VWF. VWD is classified into 6 types, type 1, type 2A, type 2B, type 2M, type 2N, and type 3. VWD frequently causes bleeding symptoms suggestive of impaired platelet aggregation (nose bleeding, purpura/subcutaneous hematoma, oral mucosal bleeding, hypermenorrhea, etc.).

(c) Acquired von Willebrand syndrome (AVWS) is a relatively rare acquired coagulopathy that assumes a pathological condition similar to that of congenital VWD in association with a pre-existing disease (lymphoproliferative disease, autoimmune disease, myeloproliferative disease, cardiovascular disease, malignant tumor, hypothyroidism, etc.), and is characterized by having bleeding diathesis neither in a previous medical history nor in a family history.

The present invention demonstrates that the object of the present invention can be achieved by providing an antibody or an antibody derivative having a binding property to ADAMTS13 and having an inhibitory effect on VWF cleaving activity, which comprises heavy chain and light chain complementarity determining regions having particular amino acid sequences.

### Target Antigen

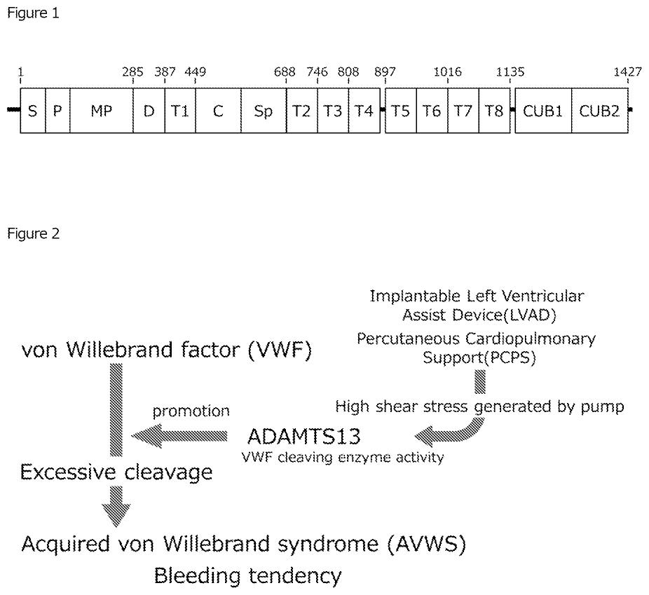

ADAMTS13 serving as a target antigen of the antibody or the antibody derivative of the present invention is an enzyme protein constituted by 1427 amino acids (GenBank No: AY055376) and has a molecular weight of approximately 200 KDa. A primary structure thereof consists of a multidomain containing a signal peptide(S), a propeptide (P), a metalloprotease domain (MP), a disintegrin-like domain (D), a thrombospondin-1 motif (T1), a cysteine-rich domain (C), a spacer domain(S), and seven repeat structures of the thrombospondin-1-motif (T2 to T8), and finally two CUB domains (CUB1 and CUB2) in order from the N-terminal side (see, FIG. 1). A physiological substrate of ADAMTS13 known in the art is only VWF, and the MDTCS domain (domain consisting of the MP domain, the D domain, the T1 domain, the C domain, and the Sp domain) of ADAMTS13 is necessary for the cleavage of VWF.

The antibody or the antibody derivative of the present invention may target any moiety in this domain structure. Examples of the antibody or the antibody derivative include: an antibody or an antibody derivative which binds to a partial amino acid sequence of the MDTCS domain as an epitope by targeting the MDTCS domain itself which is essential for the VWF cleaving activity mentioned above, wherein the antibody or the antibody derivative binds to the cleaving function domain of ADAMTS13 to block its function; and an antibody or an antibody derivative that binds to a moiety other than the MDTCS domain and conformationally prevents ADAMTS13 from binding to VWF as a result of binding to the moiety other than the MDTCS domain.

### Antibody or Antibody Derivative

In one aspect, the present invention provides an antibody or an antibody derivative thereof having a binding property to ADAMTS13 and having an inhibitory effect on VWF cleaving activity, comprising particular heavy chain and light chain complementarity determining regions (CDRs), or heavy chain and light chain variable regions comprising these CDRs or a heavy chain and a light chain comprising these CDRs.

The antibody or the antibody derivative used in the present invention is preferably free of a pathogen, such as a virus (e.g., HBV, hepatitis C virus (HCV), and human immunodeficiency virus (HIV)), which might be contained in blood (or which has the risk of contaminating blood preparations), and free of a blood-derived component having the risk of causing an immune abnormality in recipient individuals, such as an antigenic protein contained in blood or an antibody that binds to a human protein, in order to circumvent various problems (particularly, infection problems and immunity problems) peculiar to blood preparations such as blood-derived antibody preparations.

For example, the present invention can provides an antibody prepared by obtaining immortalized cells derived from antibody-producing cells under blood-derived component-free conditions and preparing the antibody from the cells under blood-derived component-free culture conditions, or a recombinant antibody that can be prepared by recombinantly expressing an antibody protein using DNA that encodes the antibody protein prepared from the immortalized cells as the antibody free of a blood-derived component.

Specifically, in one aspect, the antibody according to the present invention can be prepared by obtaining and immortalizing cell clones that produce an antibody having a binding property to ADAMTS13 from the blood of an individual administered with the whole or a portion of ADAMTS13 as a target antigen, to obtain immortalized cells derived from the antibody-producing cells, and selecting cells that produce an antibody having inhibitory activity on the VWF cleaving function of ADAMTS13 from the immortalized antibody-producing cells.

In another aspect, the present invention can also provide an antibody of recombinant type that binds to ADAMTS13 and has activity of inhibiting the VWF cleaving function of ADAMTS13 by use of a genetic recombination technique. More specifically, the present invention can also provide, as the antibody of the present invention, a monoclonal antibody having the activity of interest obtained by: in accordance with a well-known method, acquiring mRNA from immortalized antibody-producing cells selected by the method described above that produce an antibody having activity of inhibiting the VWF cleaving function of ADAMTS13, preparing cDNA from the mRNA, and obtaining a DNA sequence that encodes the antibody protein; and expressing the DNA sequence encoding the antibody in a blood-derived component-free mammalian expression system using a vector to produce a recombinant immunoglobulin (IgG).

In the present invention, a derivative of such an antibody can be used. The antibody derivative that can be used in the present invention can be, for example, but not limited to, an engineered antibody selected from the group consisting of a humanized antibody, a chimeric antibody, a single-chain antibody, a multivalent antibody, and a multispecific antibody, or a functional fragment thereof. Among them, the functional fragment that can be used is, for example, but not limited to, F(ab′)2.

Such a derivative of the antibody can be prepared in accordance with a method well known in the art after production of the antibody, and can be prepared as a recombinant antibody derivative described about the antibody of the present invention mentioned above.

The antibody or the antibody derivative prepared by the method mentioned above has a binding property to ADAMTS13. The present invention provides an antibody or an antibody derivative having inhibitory activity on the VWF cleaving function of ADAMTS13 by further screening out the antibory having the inhibitory effect among the antibodies or antibody derivatives having a binding property to ADAMTS13.

In this aspect, the particular heavy chain and light chain complementarity determining regions (CDRs) contained in the antibody or the antibody derivative of the present invention targeted to ADAMTS13 can be selected from the group consisting of the following (1) to (4):
(1) heavy chain complementarity determining regions CDR1 (GYSFTGYT, SEQ ID NO: 1), CDR2 (INPYNGGT, SEQ ID NO: 2), and CDR3 (ARTSGYLFAY, SEQ ID NO: 3), and light chain complementarity determining regions CDR1 (EDIYNR, SEQ ID NO: 4), CDR2 (GAT, SEQ ID NO: 5), and CDR3 (QQYWSSPLT, SEQ ID NO: 6);
(2) heavy chain complementarity determining regions CDR1 (GFSLPRYG, SEQ ID NO: 7), CDR2 (IWAGGST, SEQ ID NO: 8), and CDR3 (ARAGGSOPFDY, SEQ ID NO: 9), and light chain complementarity determining regions CDR1 (RDINTY, SEQ ID NO: 10), CDR2 (RAN, SEQ ID NO: 11), and CDR3 (LOYDEFPWT, SEQ ID NO: 12);
(3) heavy chain complementarity determining regions CDR1 (GFSLTRYG, SEQ ID NO: 13), CDR2 (IWAGGST, SEQ ID NO: 14), and CDR3 (ARAGGSSSFDY, SEQ ID NO: 15), and light chain complementarity determining regions CDR1 (QDINTY, SEQ ID NO: 16), CDR2 (RAN, SEQ ID NO: 17), and CDR3 (LQYDEFPWT, SEQ ID NO: 18); and
(4) heavy chain complementarity determining regions CDR1 (GFSLTGYG, SEQ ID NO: 19), CDR2 (IWADGTT, SEQ ID NO: 20), and CDR3 (ARAGGSQPFDY, SEQ ID NO: 21), and light chain complementarity determining regions CDR1 (QDINSY, SEQ ID NO: 22), CDR2 (RAN, SEQ ID NO: 23), and CDR3 (LOYDEFPWT, SEQ ID NO: 24).

In this aspect, the amino acid sequence of a particular heavy chain variable region VH domain contained in the antibody or the antibody derivative of the present invention targeted to ADAMTS13 can be selected from the group consisting of:
(1-1) the amino acid sequence of SEQ ID NO: 25, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 25,
(1-2) the amino acid sequence of SEQ ID NO: 27, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 27,
(1-3) the amino acid sequence of SEQ ID NO: 29, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 29,
(2) the amino acid sequence of SEQ ID NO: 31, or an amino acid sequence having the substitution (e.g., conservative substitution), insertion, or deletion of one or several amino acids at a moiety except for CDR1 (SEQ ID NO: 7), CDR2 (SEQ ID NO: 8), and CDR3 (SEQ ID NO: 9) in the amino acid sequence of SEQ ID NO: 31,
(3) the amino acid sequence of SEQ ID NO: 33, or an amino acid sequence having the substitution (e.g., conservative substitution), insertion, or deletion of one or several amino acids at a moiety except for CDR1 (SEQ ID NO: 13), CDR2 (SEQ ID NO: 14), and CDR3 (SEQ ID NO: 15) in the amino acid sequence of SEQ ID NO: 33, and
(4) the amino acid sequence of SEQ ID NO: 35, or an amino acid sequence having the substitution (e.g., conservative substitution), insertion, or deletion of one or several amino acids at a moiety except for CDR1 (SEQ ID NO: 19), CDR2 (SEQ ID NO: 20), and CDR3 (SEQ ID NO: 21) in the amino acid sequence of SEQ ID NO: 35.

In this case, the amino acid sequence of an existing antibody can be used as the amino acid sequence of a heavy chain constant region CH domain. For example, the amino acid sequence of a CH domain of any human antibody or an amino acid sequence engineered from such an amino acid sequence can be used. Specifically, on the basis of the technical knowledge that CDR1 to CDR3 in the amino acid sequence of each heavy chain constant region CH domain are essential for binding to a target antigen, the present invention can provide the antibody or the antibody derivative thereof as long as an amino acid sequence having the substitution (e.g., conservative substitution), insertion, or deletion of one or several amino acids at a moiety except for CDR1 to CDR3 can constitute the antibody or the antibody derivative thereof having a binding property to ADAMTS13 and having an inhibitory effect on von Willebrand factor (VWF) cleaving activity.

In this aspect, the amino acid sequence of a particular light chain variable region VL domain contained in the antibody or the antibody derivative of the present invention targeted to ADAMTS13 can be selected from the group consisting of
(1-1) the amino acid sequence of SEQ ID NO: 26, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 26,
(1-2) the amino acid sequence of SEQ ID NO: 28, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 28,
(1-3) the amino acid sequence of SEQ ID NO: 30, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 30,
(2) the amino acid sequence of SEQ ID NO: 32, or an amino acid sequence having the substitution (e.g., conservative substitution), insertion, or deletion of one or several amino acids at a moiety except for CDR1 (SEQ ID NO: 10), CDR2 (SEQ ID NO: 11), and CDR3 (SEQ ID NO: 12) in the amino acid sequence of SEQ ID NO: 32,
(3) the amino acid sequence of SEQ ID NO: 34, or an amino acid sequence having the substitution (e.g., conservative substitution), insertion, or deletion of one or several amino acids at a moiety except for CDR1 (SEQ ID NO: 16), CDR2 (SEQ ID NO: 17), and CDR3 (SEQ ID NO: 18) in the amino acid sequence of SEQ ID NO: 34, and
(4) the amino acid sequence of SEQ ID NO: 36, or an amino acid sequence having the substitution (e.g., conservative substitution), insertion, or deletion of one or several amino acids at a moiety except for CDR1 (SEQ ID NO: 22), CDR2 (SEQ ID NO: 23), and CDR3 (SEQ ID NO: 24) in the amino acid sequence of SEQ ID NO: 36.

In this case, the amino acid sequence of an existing antibody can be used as the amino acid sequence of a light chain constant region CL domain. For example, the amino acid sequence of a CL domain of any human antibody or an amino acid sequence engineered from such an amino acid sequence can be used. Specifically, on the basis of the technical knowledge that CDR1 to CDR3 in the amino acid sequence of each light chain constant region CL domain are essential for binding to a target antigen, the present invention can provide the antibody or the antibody derivative thereof as long as an amino acid sequence having the substitution (e.g., conservative substitution), insertion, or deletion of one or several amino acids at a moiety except for CDR1 to CDR3 can constitute the antibody or the antibody derivative thereof having a binding property to ADAMTS13 and having an inhibitory effect on von Willebrand factor (VWF) cleaving activity.

In this aspect, the particular heavy chain and light chain contained in the antibody or the antibody derivative of the present invention targeted to ADAMTS13 can be selected from the group consisting of the following (1-1) to (4):
(1-1) a heavy chain (the amino acid sequence of SEQ ID NO: 37, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 37) and
a light chain (the amino acid sequence of SEQ ID NO: 38, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 38);
(1-2) a heavy chain (the amino acid sequence of SEQ ID NO: 39, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 39) and
a light chain (the amino acid sequence of SEQ ID NO: 40, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 40);
(1-3) a heavy chain (the amino acid sequence of SEQ ID NO: 41, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 41) and
a light chain (the amino acid sequence of SEQ ID NO: 42, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 42);
(2) a heavy chain (the amino acid sequence of SEQ ID NO: 43, or an amino acid sequence having the substitution (e.g., conservative substitution), insertion, or deletion of one or several amino acids at a moiety except for CDR1 (SEQ ID NO: 7), CDR2 (SEQ ID NO: 8), and CDR3 (SEQ ID NO: 9) in the amino acid sequence of SEQ ID NO: 43) and
a light chain (the amino acid sequence of SEQ ID NO: 44, or an amino acid sequence having the substitution (e.g., conservative substitution), insertion, or deletion of one or several amino acids at a moiety except for CDR1 (SEQ ID NO: 10), CDR2 (SEQ ID NO: 11), and CDR3 (SEQ ID NO: 12) in the amino acid sequence of SEQ ID NO: 44);
(3) a heavy chain (the amino acid sequence of SEQ ID NO: 45, or an amino acid sequence having the substitution (e.g., conservative substitution), insertion, or deletion of one or several amino acids at a moiety except for CDR1 (SEQ ID NO: 13), CDR2 (SEQ ID NO: 14), and CDR3 (SEQ ID NO: 15) in the amino acid sequence of SEQ ID NO: 45) and
a light chain (the amino acid sequence of SEQ ID NO: 46, or an amino acid sequence having the substitution (e.g., conservative substitution), insertion, or deletion of one or several amino acids at a moiety except for CDR1 (SEQ ID NO: 16), CDR2 (SEQ ID NO: 17), and CDR3 (SEQ ID NO: 18) in the amino acid sequence of SEQ ID NO: 46); and
(4) a heavy chain (the amino acid sequence of SEQ ID NO: 47, or an amino acid sequence having the substitution (e.g., conservative substitution), insertion, or deletion of one or several amino acids at a moiety except for CDR1 (SEQ ID NO: 19), CDR2 (SEQ ID NO: 20), and CDR3 (SEQ ID NO: 21) in the amino acid sequence of SEQ ID NO: 47) and
a light chain (the amino acid sequence of SEQ ID NO: 48, or an amino acid sequence having the substitution (e.g., conservative substitution), insertion, or deletion of one or several amino acids at a moiety except for CDR1 (SEQ ID NO: 22), CDR2 (SEQ ID NO: 23), and CDR3 (SEQ ID NO: 24) in the amino acid sequence of SEQ ID NO: 48).

In this case, on the basis of the technical knowledge that, for a heavy chain and a light chain, CDR1 to CDR3 in each of the amino acid sequences of the heavy chain and the light chain are essential for binding to a target antigen, the present invention can provide the antibody or the antibody derivative thereof as long as an amino acid sequence having the substitution (e.g., conservative substitution), insertion, or deletion of one or several amino acids at a moiety except for CDR1 to CDR3 can constitute the antibody or the antibody derivative thereof having a binding property to ADAMTS13 and having an inhibitory effect on von Willebrand factor (VWF) cleaving activity.

Inhibitory effect of the antibody or the antibody derivative on VWF cleaving activity of ADAMTS13

VWF has a cleavage site by ADAMTS13 at Tyr¹⁶⁰⁵-Met¹⁶⁰⁶ of its A2 domain and assumes a structure that does not permit an access of ADAMTS13 to the cleavage site because this cleavage site is embedded within the A2 domain when it is present as a multimer in plasma. On the other hand, when a portion of the VWF multimer binds to a vascular endothelial cell, platelet, or the like and a physical force caused by blood flow or the like is applied thereto, the structure of the A2 domain is changed so that ADAMTS13 can access to the cleavage site in the A2 domain of VWF. In such a case, ADAMTS13 functions as metalloprotease that specifically cleaves the higher order structure of the multimer.

A feature of the antibody or the antibody derivative of the present invention is to have an inhibitory effect on the VWF cleaving activity of ADAMTS13 described above. Whether the antibody or the antibody derivative has an inhibitory effect on the VWF cleaving activity of ADAMTS13 can be screened by, for example, confirming by Western blot that the cleavage of VWF is inhibited. The screening by Western blot can be performed in vitro by mixing a prescribed amount of monomer VWF with a prescribed amount of ADAMTS13, adding thereto the subject antibody to be evaluated at varying concentrations, and detecting change in the amount of VWF cleaved in an antibody concentration-dependent manner by Western blot.

### Medical Use

Since a VWF multimer having a larger molecular weight has higher ability to aggregate platelet, its cleavage by ADAMTS13 (i.e., decreasing in its molecular weight) suppresses VWF-dependent platelet aggregation. Therefore, if VWF is excessively cleaved due to increased ADAMTS13 activity, platelet aggregation is excessively suppressed, causing bleeding tendency which may cause congenital VWD or acquired AVWS. On the other hand, insufficient ADAMTS13 activity may cause excessive platelet aggregation. In a living body in a normal state, the cleavage of a VWF multimer by ADAMTS13 always occurs at a constant rate, and the balance is maintained so as not to cause bleeding and not to cause abnormal blood coagulation reaction.

The antibody or the antibody derivative of the present invention can intervene in bleeding caused by the excessive cleavage of VWF due to increased ADAMTS13 activity, because the antibody or the antibody derivative has an inhibitory effect on the VWF cleaving activity of ADAMTS13, as mentioned above. Thus, in another aspect, the present invention can provide a pharmaceutical composition for preventing or treating bleeding caused by the excessive cleavage of VWF, comprising the antibody or the antibody derivative of the present invention.

The pharmaceutical composition according to this aspect may comprise one kind of antibody or antibody derivative of the present invention or plural kinds of antibodies or antibody derivatives of the present invention as an active ingredient.

This pharmaceutical composition can exert the medical use of interest in preventing or treating bleeding caused by the excessive cleavage of VWF, by the fact that the antibody or the antibody derivative of the present invention contained as an active ingredient can inhibit the cleaving activity of ADAMTS13 against VWF.

The bleeding caused by the excessive cleavage of VWF which is a symptom to be treated by application of the pharmaceutical composition of the present invention may result from a congenital etiology or an acquired etiology and is selected from the group consisting of, for example, bleeding associated with mechanical assisted circulation and anemia.

AVWS can be listed as examples of the causative disease for the bleeding associated with mechanical assisted circulation mentioned above which is a symptom to be treated by application of the pharmaceutical composition of the present invention. The pathogenesis of AVWS is as shown in FIG. 2. It is considered that the structure of a VWF multimer is changed by high shear stress generated in a pump of an apparatus when mechanical assisted circulation such as extracorporeal membrane oxygenation (ECMO), implantable left ventricular assist device (LVAD), or percutaneous cardiopulmonary support (PCPS) is used. This change of the structure of a VWF multimer is considered to trigger the excessive degradation of VWF factor by ADAMTS13.

Thus, the mechanical assisted circulation when high shear stress is generated in a pump of an apparatus is an example, but not limited to, of the mechanical assisted circulation as the cause of AVWS of the present invention to be treated by the pharmaceutical composition of the present invention. Examples of such mechanical assisted circulation can include the mechanical assisted circulation selected from the group consisting of extracorporeal membrane oxygenation (ECMO), implantable left ventricular assist device (LVAD), and percutaneous cardiopulmonary support (PCPS).

Additional use of the antibody or the antibody derivative

The antibody or the antibody derivative of the present invention can also be used for other use and can be used, for example, for detecting and/or measuring the presence or an amount of ADAMTS13 and for detecting and/or measuring the VWF cleaving activity of ADAMTS13.

For the use in detecting and/or measuring ADAMTS13, the presence or an amount of ADAMTS13 in a biological sample can be detected and/or measured by the steps of:
contacting the biological sample collected from a test subject with the antibody or the antibody derivative of the present invention in vitro; and
detecting and/or measuring ADAMTS13 in the sample bound with the antibody or the antibody derivative.

In this method, when ADAMTS13 is detected and/or measured, a detectable label is attached directly or indirectly to the antibody or the antibody derivative of the present invention, and the presence or an amount of ADAMTS13 can be detected and/or measured by use of an approach known in the art such as ELISA or Western blot.

The present invention can also provide a kit for detecting and/or measuring the presence or an amount of ADAMTS13, comprising the antibody or the antibody derivative of the present invention for detecting and/or measuring the presence or an amount of ADAMTS13 in a biological sample of a test subject through the use of the method described above. It is a feature of this kit to contain the antibody or the antibody derivative of the present invention as a primary antibody for detection in a general kit configuration that is used for performing ELISA, Western blot, or the like.

In this kit, in the case where the antibody or the antibody derivative of the present invention is indirectly detected using a label for detection attached, the kit may comprise a labeled secondary antibody for detecting the antibody or the antibody derivative of the present invention.

For the use in detecting and/or measuring the VWF cleaving activity of ADAMTS13, the VWF cleaving activity of ADAMTS13 in a biological sample can be detected and/or measured by the steps of:
adding varying concentrations of the antibody or the antibody derivative of the present invention to the biological sample collected from a test subject, followed by incubation for a given time; and
detecting and/or measuring an intact VWF multimer, an intact VWF monomer, or a cleaved VWF fragment in the sample. It is possible to determine, by the addition of the antibody or the antibody derivative of the present invention, whether the cleavage of VWF that occurs in the absence of the antibody or the antibody derivative is cleavage caused by ADAMTS13 and whether the excessive cleavage of VWF by ADAMTS13 occurs in vivo in a test subject. In this method, in the case where an intact VWF multimer, an intact VWF monomer, or a cleaved VWF fragment is detected and/or measured, the intact VWF multimer, the intact VWF monomer, or the cleaved VWF fragment can be detected and/or measured by use of an approach known in the art such as Western blot or chromatography.

The present invention can also provide a kit for detecting and/or measuring an intact VWF multimer, an intact VWF monomer, or a cleaved VWF fragment, comprising the antibody or the antibody derivative of the present invention for detecting and/or measuring the VWF cleaving activity of ADAMTS13 in a biological sample of a test subject through the use of the method described above. It is a feature of this kit to add the antibody or the antibody derivative of the present invention for inhibiting the function of ADAMTS13 to a general kit configuration that is used for performing Western blot, chromatography, or the like.

Hereinafter, the present invention will be specifically described with reference to Examples. Examples given below do not limit the present invention by any means.

### EXAMPLES

### Example 1: Preparation of Antibody

This Example was performed for obtaining an antibody against ADAMTS13 produced in a mouse as a monoclonal antibody as a result of administering ADAMTS13 as an immunogen to the mouse.

### (1) Antibody Preparation by Hybridoma Method

A mouse (BALB/cAJcl, 7 weeks old female, CLEA Japan, Inc.) was immunized by the intraperitoneal administration of 20 μg (microgram) of a full-length recombinant ADAMTS13 antigen (in-house preparation) together with 20 μg (microgram) of a Freund's complete adjuvant (manufactured by Difco Laboratories, Inc.) five times every two weeks. One months later, the ADAMTS13 antigen was boosted to the mouse. Two weeks later, blood was collected from the tail vein, and increase in an antibody titer in the blood specific for ADAMTS13 was confirmed by ELISA based on a conventional method. The ADAMTS13 antigen from the tail vein was further boosted to the mouse having an increased antibody titer for final immunization. Three days later, the spleen was recovered from the mouse.

A single cell suspension of spleen cells was prepared from the spleen in accordance with a method known in the art, which was fused with a 2-amino-6-mercaptopurine (6-thioguanine)-resistant BALB/c mouse-derived cultured myeloma cell line (P3-X63-Ag8.653; hereinafter, also referred to as X63 cells) as a parent cell line in accordance with a method generally used in the art.

Then, the obtained fused cells were cultured in a culture medium supplemented with a HAT selection solution, to conduct cloning. Two or three weeks later, clones that produced the anti-ADAMTS13 monoclonal antibody of interest were screened by ELISA using a microplate adsorbed with the ADAMTS13 antigen on a solid phase.

### (2) Screening

A culture supernatant of the hybridoma cells was used in reaction with an ADAMTS13 antigen-immobilized ELISA plate to screen the antibody of interest. In this screening, clones that specifically reacted only with the ADAMTS13 antigen were selected by removing nonspecific reactive clones that reacted with a nonspecific oligopeptide-immobilized ELISA plate.

The hybridoma cell line that specifically reacted with an ADAMTS13 antigen-immobilized ELISA plate was selected and then cloned by the limiting dilution method to obtain A10 antibody as an anti-monoclonal antibody from the hybridomas.

### Example 2: Preparation of Another Antibody

This Example was performed for obtaining another new antibody against ADAMTS13 as a monoclonal antibody, in addition to the antibody obtained in Example 1.

### (1) Antibody Preparation by Hybridoma Method

A mouse (BALB/cA, 7 weeks old female, Japan SLC, Inc.) was immunized by the footpad injection of 20 μg (microgram) of a recombinant antigen of an ADAMTS13 metalloprotease domain (MP) linked with a disintegrin-like domain (D) (MP domain+D domain; hereinafter, referred to as an “ADAMTS13 MD recombinant antigen”; in-house preparation) together with TiterMax Gold (manufactured by TiterMax USA, Inc.). Two weeks later, the antigen together with a Freund's incomplete adjuvant (manufactured by Merck KGaA) was boosted to the mouse. Three days later, blood was collected from the tail vein, and increased in a blood antibody titer specific for ADAMTS13 MD was confirmed by ELISA based on a conventional method. Popliteal lymph nodes were recovered from the mouse with an increased antibody titer.

A single cell suspension was prepared from the popliteal lymph nodes in accordance with a method known in the art, which was fused with a 2-amino-6-mercaptopurine (6-thioguanine)-resistant mouse-derived cultured myeloma cell line (P3U1 cells) as a parent cell line in accordance with a method generally used in the art.

Then, the obtained fused cells were cultured in a culture medium supplemented with a HAT selection solution, to conduct cloning. Two or three weeks later, clones that produced the anti-ADAMTS13 MD monoclonal antibody of interest were screened by ELISA using a microplate adsorbed the ADAMTS13 MD antigen on a solid phase.

### (2) Screening

A culture supernatant of the hybridoma cells was used in reaction with an ADAMTS13 MD antigen-immobilized ELISA plate to screen the antibody of interest. In this screening, clones that specifically reacted only with the ADAMTS13 MD antigen were selected by removing nonspecific reactive clones that reacted with a nonspecific protein-immobilized ELISA plate. As a result, 1G2 antibody, 12D10 antibody, and 9A7 antibody were obtained.

### Example 3: Human Chimerization of A10 Antibody

In this Example, the human chimerization of the antibody obtained in Example 1 was performed. Specifically, the heavy chain constant region (CH) and the light chain constant region (CL) of the antibody obtained in Example 1 was replaced with the heavy chain constant region (CH) and the light chain constant region (CL) of human IgG to perform the human chimerization.

### (1) Acquisition of A10 Antibody Heavy Chain Gene and Light Chain Gene

A gene encoding the A10 antibody heavy chain and a gene encoding the A10 antibody light chain (heavy chain IgG2b gene and light chain Igk gene) were cloned from the cells that produced the A10 antibody obtained in Example 1. Specifically, total RNA extracted from the A10 antibody-producing cells was used as a template to perform reverse transcription reaction using SMART CDNA Library Construction Kit (Takara Bio Inc.) capable of amplifying 5′ full-length cDNA in accordance with the attached protocol, to prepare cDNA.

The cDNA synthesized by the method described above was used as a template with KOD FX (TOYOBO Co., Ltd.) to preform PCR twice (1st PCR and 2nd PCR). In the 1st PCR, 1 μl (microlitter) of cDNA obtained by reverse transcription from each lymphoblastoid cell line (LCL) was used as a template with the following forward primer (Primer 1) and reverse primer (Primer 2) as primers to perform PCR reaction (reaction conditions: 94° C. for 2 min=>35 cycles of [98° C. for 10 sec, 55° C. for 30 sec, and 68° C. for 2 min]=>68° C. for 3 min).

TABLE 1

| | | |
|---|---|---|
| Primer 1 | AAGCAGTGGTATCAACGCAGAGT | SEQ ID NO: 49 |
| Primer 2 | taggacctgagagctttgtgggt | SEG ID NO: 50 |
| | gctgagc | |

In the 2nd PCR, 0.5 μl (microlitter) of the extension product of the 1st PCR was used as a template with the following forward primer (Primer 1) and reverse primer (Primer 2) as primers to perform PCR reaction (reaction conditions: 94° C. for 2 min=>35 cycles of [98° C. for 10 sec, 60° C. for 30 sec, and 68° C. for 2 min]=>68° C. for 3 min).

TABLE 2

| | | |
|---|---|---|
| Primer 1 | ggggcggccgcAGAGTGGC | SEQ ID NO: 51 |
| | CATTACGGCCGGG | |
| Primer 2 | GGGGAATTCtcatttaccc | SEQ ID NO: 52 |
| | ggagaccgggagatgg | |

The PCR sample was applied to QIAquick Purification Kit (Qiagen N.V.) for the removal of unreacted primers and enzyme to obtain a purified PCR product.

The purified PCR product obtained by purification was treated with a restriction enzyme, was separated and purified by agarose gel electrophoresis, and then integrated in a vector. pQEFIP vector (retrovirus vector obtained by using pQCXIP (Takara Bio Inc.) as a backbone in which CMV promoter was replaced with human EF1 alpha promoter) was used for the preparation of a vector containing the heavy chain gene, and pQEFIN vector (retrovirus vector obtained by using pQCXIN (Takara Bio Inc.) as a backbone in which CMV promoter was replaced with human EF1 alpha promoter) was used for the preparation of a vector containing the light chain gene. The PCR product was integrated in the vectors using Ligation high (TOYOBO Co., Ltd.), which was used to transform competent cells (DH-5alpha; Nippon Gene Co., Ltd.).

Plasmid was extracted from the E. coli cultured in ampicillin-containing LB liquid medium using NucleoSpin Plasmid EasyPure (MACHEREY-NAGEL GmbH & Co. KG) in accordance with the attached protocol.

### (2) Chimerization of Heavy Chain

A human IgG1 expression plasmid vector (in-house preparation) was used as a template with the following primers to perform PCR to amplify a fragment containing a human IgG1 constant region (using TOYOBO KOD plus neo) (reaction conditions: 94° C. for 2 min=>30 cycles of [98° C. for 10 sec, 58° C. for 30 sec, and 68° C. for 4.5 min]=>68° C. for 5 min).

TABLE 3

| | | |
|---|---|---|
| Primer 1 | gcctccaccaagggcccatc | SEQ ID NO: 53 |
| Primer 2 | GCggccgcggtcaccAATTC | SEQ ID NO: 54 |

The A10 antibody IgG2b heavy chain expression plasmid vector was used as a template with the following primers to perform PCR to amplify a fragment containing the variable region of the A10 antibody heavy chain (IgG2b) (using TOYOBO KOD plus neo) (reaction conditions: 94° C. for 2 min=>30 cycles of [98° C. for 10 sec, 58° C. for 30 sec, and 68° C. for 30 sec]=>68° C. for 3 min).

TABLE 4

| | | |
|---|---|---|
| Primer 1 | GAATTggtgaccgcggccGC | SEQ ID NO: 55 |
| | AGAGTGGCCATTACGG | |
| Primer 2 | gggcccttggtggaggcTGC | SEQ ID NO: 56 |
| | AGAGACAGTGACCAGA | |

Two PCR products (human IgG1 constant region and A10 antibody (IgG2b) variable region) obtained by the steps mentioned above were each treated with a restriction enzyme Dpn I (New England Biolabs Inc. (NEB)) at 37° C. for 30 minutes and were separated by agarose gel electrophoresis, followed by DNA extraction from the gel.

Two DNA fragments obtained were mixed and treated with NEBuilder HiFi DNA Assembly Master Mix (New England Biolabs Inc. (NEB)) at 50° C. for 15 minutes so that the fragments were bound with each other. This bound DNA was transformed to E. coli in accordance with a routine method, and a single colony was obtained to produce an A10 chimeric antibody (A10c antibody) heavy chain expression plasmid vector.

### (3) Chimerization of Light Chain

The human Igk expression plasmid vector (in-house preparation) was used as a template with the following primers to perform PCR to amprify a fragment containing a human Igk constant region (using TOYOBO KOD plus neo).

TABLE 5

| | | |
|---|---|---|
| Primer 1 | CGAACTGTGGCTGCACCATC | SEQ ID NO: 57 |
| Primer 2 | GCggccgcggtcaccAATTC | SEQ ID NO: 58 |

A fragment containing the variable region of the A10 antibody light chain (Igk) was amplified by PCR with the A10 antibody Igk expression plasmid vector as a template using the following primers (using TOYOBO KOD plus neo).

TABLE 6

| | | |
|---|---|---|
| Primer 1 | GAATTggtgaccgcggccGCA | SEQ ID NO: 59 |
| | GAGTGGCCATTACGG | |
| Primer 2 | gatggtgcagccacagttcgT | SEQ ID NO: 60 |
| | TTCAGCTCCAGCTTGGTCCC | |

Two PCR products (human Igk constant region and A10 antibody Igk variable region) obtained by the steps mentioned above were each treated with a restriction enzyme Dpn I (New England Biolabs Inc. (NEB)) at 37° C. for 30 minutes and were separated by agarose gel electrophoresis, followed by DNA extraction from the gel.

Two DNA fragments obtained were mixed and treated with NEBuilder HiFi DNA Assembly Master Mix (New England Biolabs Inc. (NEB)) at 50° C. for 15 minutes so that the fragments were bound with each other. This bound DNA was transformed to E. coli in accordance with a routine method, and a single colony was obtained to produce an A10 chimeric antibody (A10c antibody) light chain expression plasmid vector.

### (4) Expression of A10 Chimeric Antibody (A10c Antibody)

The A10 chimeric antibody (A10c antibody) heavy chain expression plasmid vector produced in the section (1) of this Example and the A10 chimeric antibody (A10c antibody) light chain expression plasmid vector produced in the section (2) of this Example were transiently cotransfected to Expi 293F cells (Thermo Fisher Scientific Inc.) using Expi293 Expression System (Thermo Fisher Scientific Inc.) to perform the expression and secretion of a recombinant antibody.

80 μL (microlitter) of ExpiFectamine 293 reagent and the plasmids (15 μg (microgram) each) of the heavy chain and the light chain were each mixed with 1.5 mL of Opti-MEM I (Gibco) and left standing at room temperature for 5 minutes. Then these two solutions were combined, left standing at room temperature for 20 minutes, and added to 4.5 to 5.5×10⁶ cells/mL (30 mL) of cells cultured in a 125 mL flask (Corning Inc.), followed by gyratory culture (37° C., 8% CO₂). After culture for 18 to 20 hours, 150 ML (microlitter) of ExpiFectamine 293 Transfection Enhancer 1 and 1.5 mL of ExpiFectamine 293 Transfection Enhancer 2 were added thereto, followed by gyratory culture for 5 to 6 days.

A recombinant antibody expressed by an expression system using the Expi 293F cells was purified using Protein G affinity chromatography (GE Hitrap protein GHP (1 mL)).

In this Example, A10c antibody was produced as a chimeric antibody based on the A10 antibody obtained in Example 1.

### Example 4: Humanization of Antibody

In this Example, the antibody obtained in Example 1 was humanized.

DNA sequences encoding the amino acid sequence of the heavy chain (hereinafter, referred to as the H chain) (SEQ ID NO: 37) and the amino acid sequence of the κ light chain (hereinafter, referred to as the κL chain) (SEQ ID NO: 38) of the anti-mouse ADAMTS13 antibody clone A10 (hereinafter, referred to as the mouse A10 antibody) were analyzed by alignment to a mouse antibody germline gene sequences registered in a database to identify framework regions (FRs) (four FRs, i.e., FR1 to FR4), complementarity determining regions (CDRs) (three CDRs, i.e., CDR1 to CDR3), and somatic mutation sites in the H chain and the κL chain of the mouse A10 antibody. In the H chain, CDR1 to CDR3 of the mouse A10 antibody are shown in SEQ ID NOs: 1 to 3, and the framework regions FR1 to FR4 are moieties other than CDR1 to CDR3 in the heavy chain variable region (SEQ ID NO: 25). In the κL chain, CDR1 to CDR3 are shown in SEQ ID NOs: 4 to 6, and the framework regions FR1 to FR4 are moieties other than CDR1 to CDR3 in the light chain variable region (SEQ ID NO: 26).

Subsequently, the amino acid sequences of the H chain and the κL chain of the mouse A10 antibody (SEQ ID NO: 37 and SEQ ID NO: 38, respectively) were compared with the amino acid sequences of the H chain and the κL chain of each of human tetanus antibody clone 8A7 and clone 16E8 known in the art (hereinafter, referred to as human 8A7 antibody and human 16E8 antibody, respectively) [Japanese Patent Application No. 2020-089756] to identify a site where amino acid residues were different between the mouse A10 antibody and the human antibody (human 8A7 antibody and human 16E8 antibody).

On the basis of this comparative analysis, DNA fragments were designed such that the respective CDR1 to CDR3 moieties derived from the H chain and the κL chain of the mouse A10 antibody, and amino acids at somatic mutation locations among their neighboring amino acid residues were grafted to the H chain and the κL chain of the human antibody known in the art (human 8A7 antibody and human 16E8 antibody) by use of the CDR grafting method generally known as a humanized antibody preparation technique.

A leader sequence containing a Kozak sequence (ACCATGG) was added to each of the H chain and the κL chain of the humanized A10 antibody (humanized A10h/8A7 antibody and humanized A10h/16E8 antibody). Then, a codon optimization tool (https://www.vectorbuilder.jp/tool/codon-optimization.html) was used to optimize the frequency of codon usage of the leader sequence and the variable region (FR1-CDR1-FR2-CDR2-FR3-CDR3-FR4) of the humanized antibody in order to optimize antibody molecule expression in a human cultured cell line.

The DNA sequence of the humanized A10 antibody (A10h/8A7 antibody and A10h/16E8 antibody) having been optimized in frequency of codon usage was used to optimize the frequency of codon usage such that CAI (codon adaptation index; which is a non-dimensional number that varies in the range of 0 to 1, and means that a higher numeric value represents more optimal frequency of codon usage), an index for codon optimization, was 0.9 to 1.0.

DNA sequences encoding the heavy chain (H chain) and the light chain (κL chain) of the obtained humanized A10 antibody were ligated with separate expression plasmids by the method described in Example 3 to produce a heavy chain expression plasmid vector and a light chain expression plasmid vector of the humanized A10 antibody (A10h antibody).

The humanized A10 antibody (A10h/8A7 antibody and A10h/16E8 antibody) heavy chain expression plasmid vector and the humanized A10 antibody (A10h/8A7 antibody and A10h/16E8 antibody) light chain expression plasmid vector produced in this Example were transiently cotransfected to Expi 293F cells (Thermo Fisher Scientific Inc.) using Expi293 Expression System (Thermo Fisher Scientific Inc.) to perform the expression and secretion of a recombinant antibody.

80 μL (microlitter) of ExpiFectamine 293 reagent and the plasmids (15 μg (microgram) each) of the heavy chain and the light chain were each mixed with 1.5 mL of Opti-MEM I (Gibco) and left standing at room temperature for 5 minutes. Then these two solutions were combined, left standing at room temperature for 20 minutes, and added to 4.5 to 5.5×10⁶ cells/mL (30 mL) of cells cultured in a 125 mL flask (Corning Inc.), followed by gyratory culture (37° C., 8% CO₂). After culture for 18 to 20 hours, 150 UL (microlitter) of ExpiFectamine 293 Transfection Enhancer 1 and 1.5 mL of ExpiFectamine 293 Transfection Enhancer 2 were added thereto, followed by gyratory culture for 5 to 6 days.

A recombinant antibody expressed by an expression system using the Expi 293F cells was purified using Protein G affinity chromatography (GE Hitrap protein GHP (1 mL)).

In this Example, A10h/8A7 antibody (8A7 antibody-derived) and A10h/16E8 antibody (16E8 antibody-derived) were produced as two types of humanized antibodies as to the A10 antibody produced in Example 1.

### Example 5: Amino acid sequence analysis of antibody

In this Example, the antibody prepared in Example 1, human chimerized antibody of Example 2, and humanized antibodies of Example 3 were analyzed for their amino acid sequences.

### (A) Amino Acid Sequence Analysis of Derivative of A10 Antibody (A10c Antibody and A10h/8A7 Antibody)

In the amino acid sequence analysis on the A10c antibody, the A10 chimeric antibody (A10c antibody) heavy chain expression plasmid vector and the A10 chimeric antibody (A10c antibody) light chain expression plasmid vector produced in Example 3 were analyzed for their nucleotide sequences, and the amino acid sequence of the heavy chain and the amino acid sequence of the light chain were identified on the basis of the respective DNA sequences.

In the amino acid sequence analysis on the A10h/8A7 antibody and the A10h/16E8 antibody, the A10 humanized antibody (A10h/8A7 antibody and A10h/16E8 antibody) heavy chain expression plasmid vector and the A10 humanized antibody (A10h/8A7 antibody and A10h/16E8 antibody) light chain expression plasmid vector produced in Example 4 were analyzed for their nucleotide sequences, and the amino acid sequence of the heavy chain and the amino acid sequence of the light chain were identified on the basis of the respective DNA sequences.

### (B) Amino Acid Sequence Analysis of 1G2 Antibody, 12D10 Antibody, and 9A7 Antibody

In the amino acid sequence analysis on the 1G2 antibody, the 12D10 antibody, and the 9A7 antibody produced in Example 2, total RNA extracted from the antibody-producing cells was used as a template with SMART CDNA Library Construction Kit (Takara Bio Inc.) capable of amplifying 5′ full-length cDNA in accordance with the attached protocol to prepare cDNA through reverse transcription reaction, in order to isolate heavy chain IgG1 gene or IgG2b gene and light chain Igk gene of the antibody gene (heavy chain IgG1 gene and light chain Igk gene for the 1G2 antibody; heavy chain IgG1 gene and light chain Igk gene for the 12D10 antibody; and heavy chain IgG2b gene and light chain Igk gene for the 9A7 antibody) from the cells that produced the 1G2 antibody, the 12D10 antibody, or the 9A7 antibody.

The cDNA synthesized by the method described above wase used as a template with KOD FX (TOYOBO Co., Ltd.) to preform PCR twice (1st PCR and 2nd PCR). In the 1st PCR, 1 μl (microlitter) of cDNA obtained by reverse transcription from each lymphoblastoid cell line (LCL) was used as a template with the following forward primer (Primer 1) and reverse primer (Primer 2) as primers to perform PCR reaction (reaction conditions: 94° C. for 2 min=>35 cycles of [98° C. for 10 sec, 55° C. for 30 sec, and 68° C. for 2 min]=>68° C. for 3 min).

TABLE 7

| | | |
|---|---|---|
| Primer 1 | AAGCAGTGGTATCAACGCAGAGT | SEQ ID NO: 61 |
| Primer 2 | | |
| 1G2, 12D10 | taggaccagagggctccaaggac | SEQ ID NO: 62 |
| | actggga | |
| 9A7 | taggacctgagagctttgtgggt | SEQ ID NO: 50 |
| | gctgagc | |

In the 2nd PCR, 0.5 μl (microlitter) of the extension product of the 1st PCR was used as a template with the following forward primer (Primer 1) and reverse primer (Primer 2) as primers to perform PCR reaction (reaction conditions: 94° C. for 2 min=>35 cycles of [98° C. for 10 sec, 60° C. for 30 sec, and 68° C. for 2 min]=>68° C. for 3 min).

TABLE 8

| | | |
|---|---|---|
| Primer 1 | ggggcggccgcAGAGTGGCCA | SEQ ID NO: 63 |
| | TTACGGCCGGG | |
| Primer 2 | | |
| 1G2, 12D10 | GGGGAATTCtcatttaccagg | SEQ ID NO: 64 |
| | agagtgggagaggc | |
| 9A7 | GGGGAATTCtcatttacccgg | SEQ ID NO: 52 |
| | agaccgggagatgg | |

The PCR sample was applied to QIAquick Purification Kit (Qiagen N.V.) for the removal of unreacted primers and enzyme to obtain a purified PCR product.

The purified PCR product obtained by purification was treated with a restriction enzyme, was separated and purified by agarose gel electrophoresis, and then integrated in a vector. pQEFIP vector (retrovirus vector obtained by using pQCXIP (Takara Bio Inc.) as a backbone in which CMV promoter was replaced with human EF1 alpha promoter) was used for the preparation of a vector containing the heavy chain gene, and pQEFIN vector (retrovirus vector obtained by using pQCXIN (Takara Bio Inc.) as a backbone in which CMV promoter was replaced with human EF1 alpha promoter) was used for the preparation of a vector containing the light chain gene. The PCR product was integrated in the vectors using Ligation high (TOYOBO Co., Ltd.), which was used to transform competent cells (DH-5alpha; Nippon Gene Co., Ltd.).

Plasmid was extracted from the E. coli cultured in ampicillin-containing LB liquid medium using NucleoSpin Plasmid EasyPure (MACHEREY-NAGEL GmbH & Co. KG) in accordance with the attached protocol.

The heavy chain expression plasmid vectors and the light chain expression plasmid vectors for these antibodies were analyzed for their nucleotide sequences, and the amino acid sequences of the heavy chain and the amino acid sequences of the light chain of these antibodies were identified on the basis of the respective DNA sequences.

The results of amino acid sequence analysis were as follows.

### (1-1) A10c Antibody (Chimeric A10 Antibody)

### (1-1-1) Heavy Chain

Full-length amino acid sequence of the heavy chain: SEQ ID NO: 37

Amino acid sequence of the heavy chain variable region VH domain: SEQ ID NO: 25

As a result of analyzing complementarity determining regions (CDR1, CDR2, and CDR3) in this sequence, the following was confirmed:
Heavy chain complementarity determining regions: CDR1 (GYSFTGYT, SEQ ID NO: 1), CDR2 (INPYNGGT, SEQ ID NO: 2), and CDR3 (ARTSGYLFAY, SEQ ID NO: 3).
   (1-1-2) Light chain

Full-length amino acid sequence of the light chain: SEQ ID NO: 38

Amino acid sequence of the light chain variable region VL domain: SEQ ID NO: 26

As a result of analyzing complementarity determining regions (CDR1, CDR2, and CDR3) in this sequence, the following was confirmed:
Light chain complementarity determining regions: CDR1 (EDIYNR, SEQ ID NO: 4), CDR2 (GAT, SEQ ID NO: 5), and CDR3 (QQYWSSPLT, SEQ ID NO: 6).

### (1-2) A10h/8A7 Antibody (Humanized A10 Antibody)

### (1-2-1) Heavy Chain

Full-length amino acid sequence of the heavy chain: SEQ ID NO: 39

Amino acid sequence of the heavy chain variable region VH domain: SEQ ID NO: 27

As a result of analyzing complementarity determining regions (CDR1, CDR2, and CDR3) in this sequence, the following was confirmed:
Heavy chain complementarity determining regions: CDR1 (GYSFTGYT, SEQ ID NO: 1), CDR2 (INPYNGGT, SEQ ID NO: 2), and CDR3 (ARTSGYLFAY, SEQ ID NO: 3).

### (1-2-2) Light Chain

Full-length amino acid sequence of the light chain: SEQ ID NO: 40

Amino acid sequence of the light chain variable region VL domain: SEQ ID NO: 28

As a result of analyzing complementarity determining regions (CDR1, CDR2, and CDR3) in this sequence, the following was confirmed:
Light chain complementarity determining regions: CDR1 (EDIYNR, SEQ ID NO: 4), CDR2 (GAT, SEQ ID NO: 5), and CDR3 (QQYWSSPLT, SEQ ID NO: 6).

### (1-3) A10h/16E8 Antibody (Humanized A10 Antibody)

### (1-3-1) Heavy Chain

Full-length amino acid sequence of the heavy chain: SEQ ID NO: 41

Amino acid sequence of the heavy chain variable region VH domain: SEQ ID NO: 29

As a result of analyzing complementarity determining regions (CDR1, CDR2, and CDR3) in this sequence, the following was confirmed:
Heavy chain complementarity determining regions: CDR1 (GYSFTGYT, SEQ ID NO: 1), CDR2 (INPYNGGT, SEQ ID NO: 2), and CDR3 (ARTSGYLFAY, SEQ ID NO: 3).

### (1-3-2) Light Chain

Full-length amino acid sequence of the light chain: SEQ ID NO: 42

Amino acid sequence of the light chain variable region VL domain: SEQ ID NO: 30

As a result of analyzing complementarity determining regions (CDR1, CDR2, and CDR3) in this sequence, the following was confirmed:
Light chain complementarity determining regions: CDR1 (EDIYNR, SEQ ID NO: 4), CDR2 (GAT, SEQ ID NO: 5), and CDR3 (QQYWSSPLT, SEQ ID NO: 6).

### (2) 1G2 Antibody

### (2-1) Heavy Chain

Full-length amino acid sequence of the heavy chain: SEQ ID NO: 43

Amino acid sequence of the heavy chain variable region VH domain: SEQ ID NO: 31

As a result of analyzing complementarity determining regions (CDR1, CDR2, and CDR3) in this sequence, the following was confirmed:
Heavy chain complementarity determining regions: CDR1 (GFSLPRYG, SEQ ID NO: 7), CDR2 (IWAGGST, SEQ ID NO: 8), and CDR3 (ARAGGSOPFDY, SEQ ID NO: 9).

### (2-2) Light Chain

Full-length amino acid sequence of the light chain: SEQ ID NO: 44

Amino acid sequence of the light chain variable region VL domain: SEQ ID NO: 32

As a result of analyzing complementarity determining regions (CDR1, CDR2, and CDR3) in this sequence, the following was confirmed:
Light chain complementarity determining regions: CDR1 (RDINTY, SEQ ID NO: 10), CDR2 (RAN, SEQ ID NO: 11), and CDR3 (LOYDEFPWT, SEQ ID NO: 12).

### (3) 12D10 Antibody

### (3-1) Heavy Chain

Full-length amino acid sequence of the heavy chain: SEQ ID NO: 45

Amino acid sequence of the heavy chain variable region VH domain: SEQ ID NO: 33

As a result of analyzing complementarity determining regions (CDR1, CDR2, and CDR3) in this sequence, the following was confirmed:
Heavy chain complementarity determining regions: CDR1 (GFSLTRYG, SEQ ID NO: 13), CDR2 (IWAGGST, SEQ ID NO: 14), and CDR3 (ARAGGSSSFDY, SEQ ID NO: 15).

### (3-2) Light Chain

Full-length amino acid sequence of the light chain: SEQ ID NO: 46

Amino acid sequence of the light chain variable region VL domain: SEQ ID NO: 34

As a result of analyzing complementarity determining regions (CDR1, CDR2, and CDR3) in this sequence, the following was confirmed:
Light chain complementarity determining regions: CDR1 (QDINTY, SEQ ID NO: 16), CDR2 (RAN, SEQ ID NO: 17), and CDR3 (LOYDEFPWT, SEQ ID NO: 18).

### (4) 9A7 Antibody

### (4-1) Heavy Chain

Full-length amino acid sequence of the heavy chain: SEQ ID NO: 47

Amino acid sequence of the heavy chain variable region VH domain: SEQ ID NO: 35

As a result of analyzing complementarity determining regions (CDR1, CDR2, and CDR3) in this sequence, the following was confirmed:
Heavy chain complementarity determining regions: CDR1 (GFSLTGYG, SEQ ID NO: 19), CDR2 (IWADGTT, SEQ ID NO: 20), and CDR3 (ARAGGSOPFDY, SEQ ID NO: 21).
   (4-2) Light chain

Full-length amino acid sequence of the light chain: SEQ ID NO: 48

Amino acid sequence of the light chain variable region VL domain: SEQ ID NO: 36

As a result of analyzing complementarity determining regions (CDR1, CDR2, and CDR3) in this sequence, the following was confirmed:
Light chain complementarity determining regions: CDR1 (QDINSY, SEQ ID NO: 22), CDR2 (RAN, SEQ ID NO: 23), and CDR3 (LOYDEFPWT, SEQ ID NO: 24).

### Example 6: In Vitro Binding Property Analysis of Antibodies

In this Example, a binding property to the target molecule ADAMTS13 among chimeric antibodies of various types using the heavy chain and the light chain of each antibody prepared in Example 1 and Example 2 and the heavy chain and the light chain of each human chimerized antibody prepared in Example 3 were compared and studied for change in a binding property by chimerization of various types of antibodies.

The antibodies used in this Example were recombinant A10 antibody consisting of the heavy chain or the light chain of the mouse A10 antibody, and recombinant human chimeric antibody (A10c antibody) consisting of the heavy chain or the light chain of the A10c antibody prepared in Example 3.

A antibody for detection for use in detecting each antibody (A10c antibody and A10 antibody) was an anti-human IgG antibody for the A10c antibody and an anti-mouse IgG antibody for the A10 antibody which were each used as a “secondary antibody”.

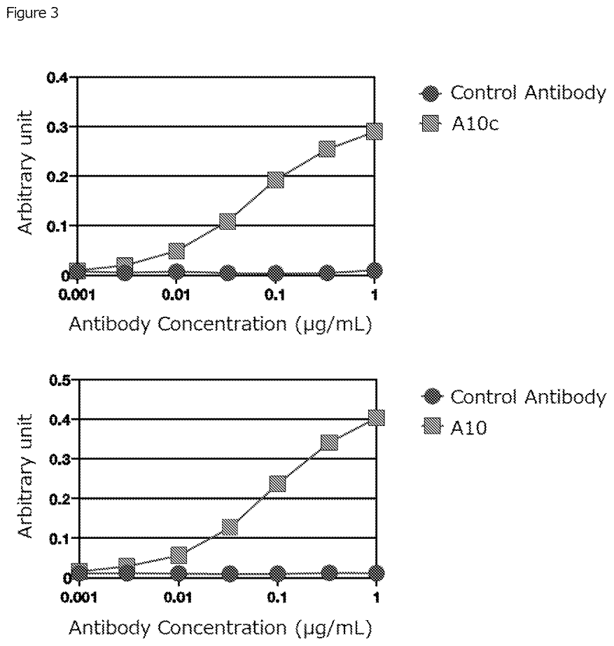

The binding property was measured by ELISA using wells on which 50 ng of the ADAMTS13 MD recombinant antigen (in-house preparation) was immobilized. Specifically, ADAMTS13 MD was diluted into 1 μg (microgram)/mL with PBS, added at 50 μL (microlitter)/well to a 96-well plate for ELISA (NUNC), and left standing overnight at 4° C. The plate was washed three times (250 μL (microlitter)/well of PBS-T (0.1% Tween 20 in PBS)), and after addition of 250 μL (microlitter)/well of a blocking buffer (2% BSA, 0.05% NaN3 in PBS), left standing at room temperature for 1 hour. Then the 96-well plate was washed once (250 μL (microlitter)/well of PBS-T). Each recombinant antibody (A10c antibody and A10 antibody) was concentration-adjusted and then 50 μL (microlitter)/well of recombinant antibody was added to each well of the plate, which was left standing at room temperature for 2 hours. Each recombinant antibody was used at concentrations of 1×10⁻μg (microgram)/mL, 5×10⁻³ μg (microgram)/mL, 1×10−2 μg (microgram)/mL, 5×10⁻² μg (microgram)/mL, 1×10⁻¹ μg (microgram)/mL, 5×10⁻¹ μg (microgram)/mL, and 1 μg (microgram)/mL, as also shown in FIG. 3.

Then the 96-well plate was washed three times (250 μL (microlitter)/well of PBS-T). An alkaline phosphatase-labeled secondary antibody (goat anti-human IgG-AP and goat anti-mouse IgG-AP (SouthernBiotech)) was diluted 1/1000-fold with PBS-T and added at 50 ML (microlitter)/well, and the plate was left standing at room temperature for 1 hour. Then the 96-well plate was washed once (250 μL (microlitter)/well of PBS-T). Then, a chromogenic substrate solution containing one particle of a phosphatase substrate (Sigma) dissolved in 25 mL of 0.1 M glycine buffer was added at 100 μL (microlitter)/well. Absorbance at 405 nm and 650 nm was measured using multimode reader EnSpire (PerkinElmer Enspire).

As a result, both the tested antibodies (A10c antibody and A10 antibody) bound to ADAMTS13. The binding strength of the engineered antibodies to ADAMTS13 was not altered by chimerization (in A10c antibody), and dose-dependent reactivity with the antigen was shown, as in the A10 antibody (see, FIG. 3).

Example 7: In vitro inhibitory effect analysis of antibodies

In this Example, the A10 antibody prepared in Example 1 and the 1G2 antibody, the 12D10 antibody, and the 9A7 antibody prepared in Example 2 were analyzed for their inhibitory effects on the VWF cleaving activity of the target molecule ADAMTS13.

20 μL (microlitter) of 0.2 to 200 μg (microgram)/mL each antibody (A10 antibody produced in Example 1 and 1G2 antibody, 12D10 antibody, and 9A7 antibody produced in Example 2) were mixed with 50 μL (microlitter) of normal human plasma to adjust a final concentration to 0.01 to 100 μg (microgram)/mL. Then, the mixture was incubated at 37° C. for 15 minutes, followed by the measurement of ADAMTS13 activity remaining in the human plasma by ELISA (KAINOS Laboratories, Inc.).

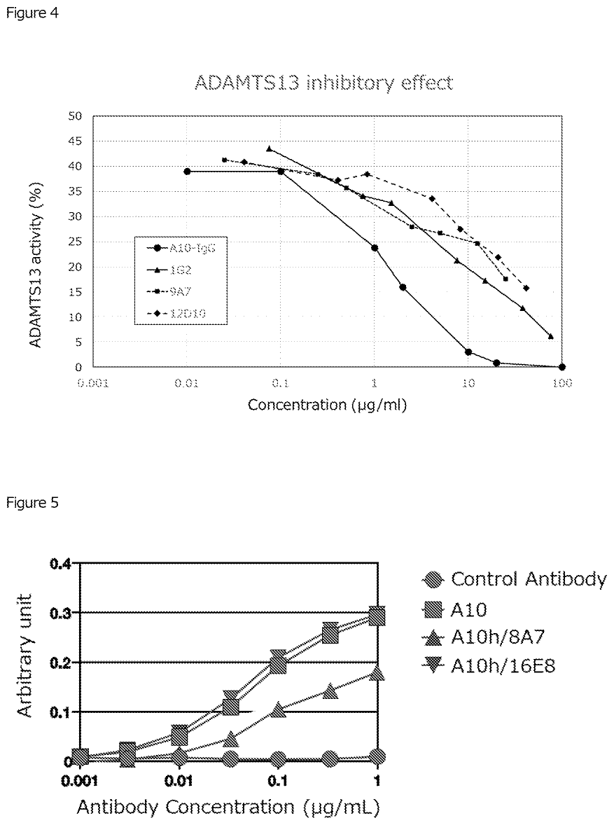

As a result, the A10 antibody exhibited an inhibitory effect with an ID50 concentration of approximately 1.55 μg (microgram)/mL on the VWF cleaving activity of ADAMTS13 and exhibited a complete (almost 100%) inhibitory effect at 50 μg (microgram)/mL or higher (see, FIG. 4).

The 1G2 antibody exhibited an inhibitory effect with an ID50 concentration of approximately 7.28 μg (microgram)/mL on the VWF cleaving activity of ADAMTS13, the 12D10 antibody exhibited an inhibitory effect with an ID50 concentration of approximately 25.4 μg (microgram)/mL on the VWF cleaving activity of ADAMTS13, and the 9A7 antibody exhibited an inhibitory effect with an ID50 concentration of approximately 19.5 μg (microgram)/mL on the VWF cleaving activity of ADAMTS13 (see, FIG. 4).

### Example 8: In Vitro Functional Analysis of Humanized Antibody

In this Example, a binding property to and inhibitory activity against the target molecule ADAMTS13 were compared and studied for change by humanization in its function (binding property and inhibitory activity) of humanized antibody prepared in Example 4 (A10h/8A7 antibody and A10h/16E8 antibody) when compared with the human chimerized antibody of Example 3.

The binding property analysis was conducted in the same manner as in Example 6 except that a plurality of concentrations of the target molecule ADAMTS13 were set. The goat anti-human IgG-AP was used as a secondary antibody.

As a result, the tested A10h/16E8 antibody (the humanized antibody of the A10 antibody) exhibited a binding property to ADAMTS13 at almost the same level as that of the A10c antibody (the human chimeric antibody of the A10 antibody) with its binding property confirmed in Example 6 (see, FIG. 5). On the other hand, the A10h/8A7 antibody (the humanized antibody of the A10 antibody) was found to maintain binding strength on the order of 60% of the binding strength of the A10c antibody (the human chimeric antibody of the A10 antibody), though the binding property to ADAMTS13 was reduced as compared with the A10c antibody (see, FIG. 5). These results demonstrated that the A10 antibody, even if humanized, exhibits dose-dependent reactivity with the ADAMTS13 antigen, as in the chimeric antibody (A10c antibody).

Subsequently, the inhibitory activity of each antibody (A10h/8A7 antibody, A10h/16E8 antibody, A10 antibody, and A10c antibody) was measured in the same manner as in Example 7.

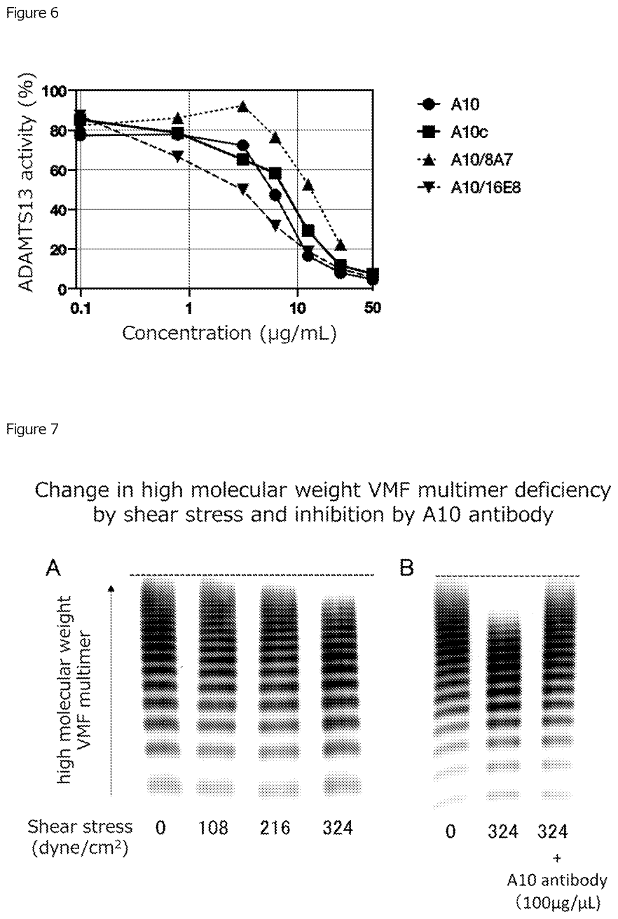

As a result, the tested A10h/16E8 antibody (the humanized antibody of the A10 antibody) exhibited, in a dose-dependent manner, higher inhibitory activity against ADAMTS13 than that of the A10 antibody with its inhibitory activity confirmed in Example 7 (see, FIG. 6). On the other hand, the A10h/8A7 antibody (the humanized antibody of the A10 antibody) exhibited dose-dependent inhibitory activity against ADAMTS13, as in the A10h/16E8 antibody, though the inhibitory activity against ADAMTS13 was reduced as compared with the A10 antibody (see, FIG. 6). The A10 antibody and the A10c antibody (the human chimeric antibody of the A10 antibody) were found to have almost the same level of inhibitory activity (see, FIG. 6). These results demonstrated that the A10 antibody, even if humanized, exhibits dose-dependent inhibitory activity against the ADAMTS13 antigen, as in the chimeric antibody (A10c antibody).

In this Example, the ID50 value of each antibody against the VWF cleaving activity of ADAMTS13 was calculated. As a result, each antibody was found to have the following ID50 value (see, FIG. 6):
A10 antibody=5.97 μg (microgram)/mL;
A10c antibody=6.92 μg (microgram)/mL;
A10h/8A7 antibody=13.04 μg (microgram)/mL; and
A10h/16E8 antibody=2.52 μg (microgram)/mL.

### Example 9: In vitro inhibitory effect analysis of antibodies

In this Example, the cleavage of a VWF multimer that occurred in blood under high shear stress applied to the blood was varidated to confirm how the antibodies against ADAMTS13 of the present invention had an effect on the cleavage of a VWF multimer.

First, human plasma was loaded with high shear stress, and VWF cleavage by ADAMTS13 was confirmed by VWF multimer analysis. The high shear stress was loaded by placing plasma in two 1 mL syringes or two 2.5 mL syringes (both from TERUMO Corp.) connected through an 18-G injection needle (TERUMO Corp.), and moving the plasma between the syringes once every second for 6 minutes (360 times) so that the plasma passed through the injection needle (see, J Thromb Haemost, 2019; 17:975-983). In this operation, high shear stress of 108 dyne/cm², 216 dyne/cm², and 324 dyne/cm² were generated at plasma volumes of 440 UL (microlitter), 880 UL (microlitter), and 1320 UL (microlitter), respectively. The high shear stress of 324 dyne/cm² is very high shear stress that is not observed in the body of a healthy person.

In the VWF multimer analysis, the plasma was electrophoresed on SDS-1.0% agarose gel and visualized by Western blot. The antibody for detection used was VWF polyclonal antibody (Dako).

As a result, more deficiency in high-molecular-weight VWF multimer and stronger cleavage of VWF were shown with increase in shear stress of 108 dyne/cm², 216 dyne/cm², and 324 dyne/cm² (see, FIG. 7A).

Next, the A10 antibody prepared in Example 1 was added at 100 μg (microgram)/mL to the plasma. After 15 minutes, high shear stress of 324 dyne/cm² was generated in the same manner as above, and the cleavage of a VWF multimer was analyzed by the same method as above.

As a result, the absence of VWF cleavage by the addition of the anti-ADAMTS13 antibody was able to be observed even when loaded with high shear stress of 324 dyne/cm² (see, FIG. 7B). This suggested that the anti-ADAMTS13 antibody can prevent the development of AVWS caused by the excessive cleavage of VWF by ADAMTS13 under abnormally high shear stress.

### INDUSTRIAL APPLICABILITY

The present invention can provide an antibody or an antibody derivative that has specific binding activity against ADAMTS13, VWF cleaving protease, which is considered as a causative factor of the acquired von Willebrand syndrome (AVWS) in humans, and can reduce the excessive cleavage of VWF by ADAMTS13. Since the excessive cleavage of VWF by ADAMTS13 is considered as pathogenesis of the human acquired von Willebrand syndrome (AVWS), the present invention can also provide a pharmaceutical composition for preventing or treating human AVWS, comprising the antibody or the antibody derivative of the present invention.

### Free Text of Sequence Listing

Heavy chain complementarity determining regions of A10c antibody, A10h/8A7 antibody, and A10h/16E8 antibody: CDR1 (GYSFTGYT, SEQ ID NO: 1), CDR2 (INPYNGGT, SEQ ID NO: 2), and CDR3 (ARTSGYLFAY, SEQ ID NO: 3)
Light chain complementarity determining regions of A10c antibody, A10h/8A7 antibody, A10h/16E8 antibody: CDR1 (EDIYNR, SEQ ID NO: 4), CDR2 (GAT, SEQ ID NO: 5), and CDR3 (QQYWSSPLT, SEQ ID NO: 6) 1G2 antibody Heavy chain complementarity determining regions: CDR1 (GFSLPRYG, SEQ ID NO: 7), CDR2 (IWAGGST, SEQ ID NO: 8), and CDR3 (ARAGGSOPFDY, SEQ ID NO: 9)
1G2 antibody Light chain complementarity determining regions: CDR1 (RDINTY, SEQ ID NO: 10), CDR2 (RAN, SEQ ID NO: 11), and CDR3 (LOYDEFPWT, SEQ ID NO: 12)
12D10 antibody Heavy chain complementarity determining regions: CDR1 (GFSLTRYG, SEQ ID NO: 13), CDR2 (IWAGGST, SEQ ID NO: 14), and CDR3 (ARAGGSSSFDY, SEQ ID NO: 15)
12D10 antibody Light chain complementarity determining regions: CDR1 (QDINTY, SEQ ID NO: 16), CDR2 (RAN, SEQ ID NO: 17), and CDR3 (LOYDEFPWT, SEQ ID NO: 18)
9A7 antibody Heavy chain complementarity determining regions: CDR1 (GFSLTGYG, SEQ ID NO: 19), CDR2 (IWADGTT, SEQ ID NO: 20), and CDR3 (ARAGGSQPFDY, SEQ ID NO: 21)
9A7 antibody Light chain complementarity determining regions: CDR1 (QDINSY, SEQ ID NO: 22), CDR2 (RAN, SEQ ID NO: 23), and CDR3 (LOYDEFPWT, SEQ ID NO: 24)
Amino acid sequence of A10c antibody heavy chain variable region VH domain: SEQ ID NO: 25
Amino acid sequence of A10c antibody light chain variable region VL domain: SEQ ID NO: 26
Amino acid sequence of A10h/8A7 antibody heavy chain variable region VH domain: SEQ ID NO: 27
Amino acid sequence of A10h/8A7 antibody light chain variable region VL domain: SEQ ID NO: 28
Amino acid sequence of A10h/16E8 antibody heavy chain variable region VH domain: SEQ ID NO: 29
Amino acid sequence of A10h/16E8 antibody light chain variable region VL domain: SEQ ID NO: 30
Amino acid sequence of 1G2 antibody heavy chain variable region VH domain: SEQ ID NO: 31
Amino acid sequence of 1G2 antibody light chain variable region VL domain: SEQ ID NO: 32 Amino acid sequence of 12D10 antibody heavy chain variable region VH domain: SEQ ID NO: 33
Amino acid sequence of 12D10 antibody light chain variable region VL domain: SEQ ID NO: 34
Amino acid sequence of 9A7 antibody heavy chain variable region VH domain: SEQ ID NO: 35
Amino acid sequence of 9A7 antibody light chain variable region VL domain: SEQ ID NO: 36
Full-length amino acid sequence of A10c antibody heavy chain: SEQ ID NO: 37
Full-length amino acid sequence of A10c antibody light chain: SEQ ID NO: 38
Full-length amino acid sequence of A10h/8A7 antibody heavy chain: SEQ ID NO: 39
Full-length amino acid sequence of A10h/8A7 antibody light chain: SEQ ID NO: 40
Full-length amino acid sequence of A10h/16E8 antibody heavy chain: SEQ ID NO: 41
Full-length amino acid sequence of A10h/16E8 antibody light chain: SEQ ID NO: 42
Full-length amino acid sequence of 1G2 antibody heavy chain: SEQ ID NO: 43
Full-length amino acid sequence of 1G2 antibody light chain: SEQ ID NO: 44
Full-length amino acid sequence of 12D10 antibody heavy chain: SEQ ID NO: 45
Full-length amino acid sequence of 12D10 antibody light chain: SEQ ID NO: 46
Full-length amino acid sequence of 9A7 antibody heavy chain: SEQ ID NO: 47
Full-length amino acid sequence of 9A7 antibody light chain: SEQ ID NO: 48
Primer sequences used in Example 5: SEQ ID NO: 49 to SEQ ID NO: 64

The invention claimed is:

1. An antibody or an antibody derivative thereof capable of binding to ADAMTS13 and having an inhibitory effect on von Willebrand factor (VWF) cleaving activity, comprising heavy chain and light chain complementarity determining regions selected from the group consisting of:
(1) heavy chain complementarity determining regions CDR1 (GYSFTGYT, SEQ ID NO: 1, CDR2 (INPYNGGT, SEQ ID NO: 2), and CDR3 (ARTSGYLFAY, SEQ ID NO: 3), and light chain complementarity determining regions CDR1 (EDIYNR, SEQ ID NO: 4), CDR2 (GAT, SEQ ID NO: 5), and CDR3 (QQYWSSPLT, SEQ ID NO: 6);
(2) heavy chain complementarity determining regions CDR1 (GFSLPRYG, SEQ ID NO: 7, CDR2 (IWAGGST, SEQ ID NO: 8), and CDR3 (ARAGGSQPFDY, SEQ ID NO: 9), and light chain complementarity determining regions CDR1 (RDINTY, SEQ ID NO: 10), CDR2 (RAN, SEQ ID NO: 11), and CDR3 (LQYDEFPWT, SEQ ID NO: 12);
(3) heavy chain complementarity determining regions CDR1 (GFSLTRYG, SEQ ID NO: 13, CDR2 (IWAGGST, SEQ ID NO: 14), and CDR3 (ARAGGSSSFDY, SEQ ID NO: 15), and
light chain complementarity determining regions CDR1 (QDINTY, SEQ ID NO: 16), CDR2 (RAN, SEQ ID NO: 17), and CDR3 (LQYDEFPWT, SEQ ID NO: 18); and
(4) heavy chain complementarity determining regions CDR1 (GFSLTGYG, SEQ ID NO: 19, CDR2 (IWADGTT, SEQ ID NO: 20), and CDR3 (ARAGGSQPFDY, SEQ ID NO: 21), and light chain complementarity determining regions CDR1 (QDINSY, SEQ ID NO: 22), CDR2 (RAN, SEQ ID NO: 23), and CDR3 (LQYDEFPWT, SEQ ID NO: 24).

2. The antibody or the antibody derivative according to claim 1, wherein the antibody or the antibody derivative is a recombinant type.

3. The antibody or the antibody derivative according to claim 1, wherein the antibody derivative is selected from the group consisting of an engineered antibody, a humanized antibody, a chimeric antibody, a single-chain antibody, a multivalent antibody, a multispecific antibody, and a functional fragment thereof.

4. The antibody or the antibody derivative according to claim 1, wherein an amino acid sequence of a heavy chain variable region VH domain of the antibody or the antibody derivative is selected from the group consisting of:
(1-1) the amino acid sequence of SEQ ID NO: 25, or an amino acid sequence having a substitution, insertion, or deletion of one or several amino acids at a moiety except for CDR1 (SEQ ID NO: 1), CDR2 (SEQ ID NO: 2), and CDR3 (SEQ ID NO: 3 in the amino acid sequence of SEQ ID NO: 25,
(1-2) the amino acid sequence of SEQ ID NO: 27, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3 in the amino acid sequence of SEQ ID NO: 27,
(1-3) the amino acid sequence of SEQ ID NO: 29, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3 in the amino acid sequence of SEQ ID NO: 29,
(2) the amino acid sequence of SEQ ID NO: 31, or an amino acid sequence having the substitution (e.g., conservative substitution), insertion, or deletion of one or several amino acids at a moiety except for CDR1 (SEQ ID NO: 7), CDR2 (SEQ ID NO: 8), and CDR3 (SEQ ID NO: 9 in the amino acid sequence of SEQ ID NO: 31,
(3) the amino acid sequence of SEQ ID NO: 33, or an amino acid sequence having the substitution (e.g., conservative substitution), insertion, or deletion of one or several amino acids at a moiety except for CDR1 (SEQ ID NO: 13), CDR2 (SEQ ID NO: 14), and CDR3 (SEQ ID NO: 15) in the amino acid sequence of SEQ ID NO: 33, and
(4) the amino acid sequence of SEQ ID NO: 35, or an amino acid sequence having the substitution (e.g., conservative substitution), insertion, or deletion of one or several amino acids at a moiety except for CDR1 (SEQ ID NO: 19), CDR2 (SEQ ID NO: 20), and CDR3 (SEQ ID NO: 21) in the amino acid sequence of SEQ ID NO: 35.

5. The antibody or the antibody derivative according to claim 1, wherein an amino acid sequence of a light chain variable region VL domain of the antibody or the antibody derivative is selected from the group consisting of:
(1-1) the amino acid sequence of SEQ ID NO: 26, or an amino acid sequence having a substitution, insertion, or deletion of one or several amino acids at a moiety except for CDR1 (SEQ ID NO: 4), CDR2 (SEQ ID NO: 5), and CDR3 (SEQ ID NO: 6 in the amino acid sequence of SEQ ID NO: 26,
(1-2) the amino acid sequence of SEQ ID NO: 28, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6 in the amino acid sequence of SEQ ID NO: 28,
(1-3) the amino acid sequence of SEQ ID NO: 30, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6 in the amino acid sequence of SEQ ID NO: 30,
(2) the amino acid sequence of SEQ ID NO: 32, or an amino acid sequence having the substitution (e.g., conservative substitution), insertion, or deletion of one or several amino acids at a moiety except for CDR1 (SEQ ID NO: 10), CDR2 (SEQ ID NO: 11), and CDR3 (SEQ ID NO: 12) in the amino acid sequence of SEQ ID NO: 32,
(3) the amino acid sequence of SEQ ID NO: 34, or an amino acid sequence having the substitution (e.g., conservative substitution), insertion, or deletion of one or several amino acids at a moiety except for CDR1 (SEQ ID NO: 16), CDR2 (SEQ ID NO: 17), and CDR3 (SEQ ID NO: 18) in the amino acid sequence of SEQ ID NO: 34, and
(4) the amino acid sequence of SEQ ID NO: 36, or an amino acid sequence having the substitution (e.g., conservative substitution), insertion, or deletion of one or several amino acids at a moiety except for CDR1 (SEQ ID NO: 22), CDR2 (SEQ ID NO: 23), and CDR3 (SEQ ID NO: 24) in the amino acid sequence of SEQ ID NO: 36.

6. The antibody or the antibody derivative according to claim 1, wherein the antibody or the antibody derivative is selected from the group consisting of:
(1-1) an antibody or an antibody derivative comprising a heavy chain (the amino acid sequence of SEQ ID NO: 37, or an amino acid sequence having a substitution, insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 37) and
a light chain (the amino acid sequence of SEQ ID NO: 38, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 38);
(1-2) a heavy chain (the amino acid sequence of SEQ ID NO: 39, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 39) and
a light chain (the amino acid sequence of SEQ ID NO: 40, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 40);
(1-3) a heavy chain (the amino acid sequence of SEQ ID NO: 41, or an amino acid sequence having the substitution (e.g., conservative substitution), insertion, or deletion of one or several amino acids at a moiety except for CDR1 (SEQ ID NO: 1), CDR2 (SEQ ID NO: 2), and CDR3 (SEQ ID NO: 3) in the amino acid sequence of SEQ ID NO: 41) and
a light chain (the amino acid sequence of SEQ ID NO: 42, or an amino acid sequence having the substitution (e.g., conservative substitution), insertion, or deletion of one or several amino acids at a moiety except for CDR1 (SEQ ID NO: 4), CDR2 (SEQ ID NO: 5), and CDR3 (SEQ ID NO: 6) in the amino acid sequence of SEQ ID NO: 42);
(2) an antibody or an antibody derivative comprising
a heavy chain (the amino acid sequence of SEQ ID NO: 43, or an amino acid sequence having the substitution (e.g., conservative substitution), insertion, or deletion of one or several amino acids at a moiety except for CDR1 (SEQ ID NO: 7), CDR2 (SEQ ID NO: 8), and CDR3 (SEQ ID NO: 9) in the amino acid sequence of SEQ ID NO: 43) and
a light chain (the amino acid sequence of SEQ ID NO: 44, or an amino acid sequence having the substitution (e.g., conservative substitution), insertion, or deletion of one or several amino acids at a moiety except for CDR1 (SEQ ID NO: 10), CDR2 (SEQ ID NO: 11), and CDR3 (SEQ ID NO: 12) in the amino acid sequence of SEQ ID NO: 44);
(3) an antibody or an antibody derivative comprising
a heavy chain (the amino acid sequence of SEQ ID NO: 45, or an amino acid sequence having the substitution (e.g., conservative substitution), insertion, or deletion of one or several amino acids at a moiety except for CDR1 (SEQ ID NO: 13), CDR2 (SEQ ID NO: 14), and CDR3 (SEQ ID NO: 15) in the amino acid sequence of SEQ ID NO: 45) and
a light chain (the amino acid sequence of SEQ ID NO: 46, or an amino acid sequence having the substitution (e.g., conservative substitution), insertion, or deletion of one or several amino acids at a moiety except for CDR1 (SEQ ID NO: 16), CDR2 (SEQ ID NO: 17), and CDR3 (SEQ ID NO: 18) in the amino acid sequence of SEQ ID NO: 46); and
(4) a heavy chain (the amino acid sequence of SEQ ID NO: 47, or an amino acid sequence having the substitution (e.g., conservative substitution), insertion, or deletion of one or several amino acids at a moiety except for CDR1 (SEQ ID NO: 19), CDR2 (SEQ ID NO: 20), and CDR3 (SEQ ID NO: 21) in the amino acid sequence of SEQ ID NO: 47) and
a light chain (the amino acid sequence of SEQ ID NO: 48, or an amino acid sequence having the substitution (e.g., conservative substitution), insertion, or deletion of one or several amino acids at a moiety except for CDR1 (SEQ ID NO: 22), CDR2 (SEQ ID NO: 23), and CDR3 (SEQ ID NO: 24) in the amino acid sequence of SEQ ID NO: 48).

7. A pharmaceutical composition for preventing or treating bleeding caused by the excessive cleavage of VWF, comprising the antibody or the antibody derivative according to any claim 1.

8. The pharmaceutical composition according to claim 7, wherein the bleeding caused by the excessive cleavage of VWF is bleeding associated with mechanical assisted circulation.

9. (canceled)

10. The pharmaceutical composition according to claim 7, wherein the pharmaceutical composition inhibits the cleaving activity of ADAMTS13 against VWF.

11. The pharmaceutical composition according to claim 7, wherein the bleeding associated with mechanical assisted circulation is acquired von Willebrand syndrome (AVWS).

12. The pharmaceutical composition according to claim 7, wherein the mechanical assisted circulation is selected from the group consisting of extracorporeal membrane oxygenation (ECMO), implantable left ventricular assist device (LVAD), and percutaneous cardiopulmonary support (PCPS).

13. A method for detecting and/or measuring the presence or an amount of ADAMTS13 in a biological sample, comprising:
contacting the biological sample collected from a test subject with the antibody or the antibody derivative according to claim 1 in vitro; and
detecting and/or measuring ADAMTS13 in the sample bound with the antibody or the antibody derivative.

14. A kit for detecting and/or measuring the presence or an amount of ADAMTS13 in the body of a test subject, comprising the antibody or the antibody derivative according to claim 1.

15. A method for detecting and/or measuring the VWF cleaving activity of ADAMTS13 in a biological sample, comprising:
adding varying concentrations of the antibody or the antibody derivative according to claim 1 to the biological sample collected from a test subject, followed by incubation; and
detecting and/or measuring an intact VWF multimer, an intact VWF monomer, or a cleaved VWF fragment in the sample.

16. A kit for detecting and/or measuring an intact VWF multimer, an intact VWF monomer, or a cleaved VWF fragment in the body of a test subject, comprising the antibody or the antibody derivative according to claim 1.


SEQUENCE LISTING (64 sequences)

SEQ ID NO: 1 - protein, 8 aa, Mus musculus
GYSFTGYT

SEQ ID NO: 2 - protein, 8 aa, Mus musculus
INPYNGGT

SEQ ID NO: 3 - protein, 10 aa, Mus musculus
ARTSGYLFAY

SEQ ID NO: 4 - protein, 6 aa, Mus musculus
EDIYNR

SEQ ID NO: 5 - skipped

SEQ ID NO: 6 - protein, 9 aa, Mus musculus
QQYWSSPLT

SEQ ID NO: 7 - protein, 8 aa, Mus musculus
GFSLPRYG

SEQ ID NO: 8 - protein, 7 aa, Mus musculus
IWAGGST

SEQ ID NO: 9 - protein, 11 aa, Mus musculus
ARAGGSQPFDY

SEQ ID NO: 10 - protein, 6 aa, Mus musculus
RDINTY

SEQ ID NO: 11 - skipped

SEQ ID NO: 12 - protein, 9 aa, Mus musculus
LQYDEFPWT

SEQ ID NO: 13 - protein, 8 aa, Mus musculus
GFSLTRYG

SEQ ID NO: 14 - protein, 7 aa, Mus musculus
IWAGGST

SEQ ID NO: 15 - protein, 11 aa, Mus musculus
ARAGGSSSFDY

SEQ ID NO: 16 - protein, 6 aa, Mus musculus
QDINTY

SEQ ID NO: 17 - skipped

SEQ ID NO: 18 - protein, 9 aa, Mus musculus
LQYDEFPWT

SEQ ID NO: 19 - protein, 8 aa, Mus musculus
GFSLTGYG

SEQ ID NO: 20 - protein, 7 aa, Mus musculus
IWADGTT

SEQ ID NO: 21 - protein, 11 aa, Mus musculus
ARAGGSQPFDY

SEQ ID NO: 22 - protein, 6 aa, Mus musculus
QDINSY

SEQ ID NO: 23 - skipped

SEQ ID NO: 24 - protein, 9 aa, Mus musculus
LQYDEFPWT

SEQ ID NO: 25 - protein, 117 aa, Mus musculus
EVQLQQSGPELVKPGTSMKISCKASGYSFTGYTMHWVKQSHGKNLEWIGLINPYNGGTGYNQKFKGKATLTVDKSSSTTYIELLSLTSEDSAVYYCARTSGYLFAYWGQGTLVTVSA

SEQ ID NO: 26 - protein, 107 aa, Mus musculus
DIQMTQSSSSFSVSLGDRVTITCKASEDIYNRLAWYQQKPGNAPRLLISGATSLETGVPSRFSGSGSGKDYIFSITSLQTEDVATYYCQQYWSSPLTFGAGTKLELK

SEQ ID NO: 27 - protein, 117 aa, Unknown
  Humanized
QIHLVESGGGVVQTGKSMKISCKASGYSFTGYTMHWVKQTPGKNLEWIGLINPYNGGTGYNQKFKGKATLTVDKSSSTTYIELLSLTSEDSAVYYCARTSGYLFAYWGQGTLVTVSS

SEQ ID NO: 28 - protein, 107 aa, Unknown
  Humanized
ETVMTQSPATLSVSPGERATITCKASEDIYNRLAWYQQKPGQAPRLLISGATSLETGVPSRFSGSGSGTEFTLTISSLQSEDVATYYCQQYWSSPLTFGAGTKLEIK

SEQ ID NO: 29 - protein, 117 aa, Unknown
  Humanized
QVQLVQSGGQVKKPGASVKISCKASGYSFTGYTMHWVKQAPGRGLEWIGLINPYNGGTGYNQKFKGKATLTVDKSSSTTYIELLSLTSEDSAVYYCARTSGYLFAYWGQGTLVTVSS

SEQ ID NO: 30 - protein, 107 aa, Unknown
  Humanized
DIVLTQTPDSLAVSLGERATITCKASEDIYNRLAWYQQKAGQPPKLLISGATSLETGVPDRFSGSGSGTDFTLTISSLQAEDVATYYCQQYWSSPLTFGAGTKVNIK

SEQ ID NO: 31 - protein, 117 aa, Mus musculus
QVQLRESGPGLVAPSQSLSITCTVSGFSLPRYGIHWVRQPPGKGLEWLGVIWAGGSTDYDSALMSRLSISKDTSKSQVFLKMSSLQTDDTAIYYCARAGGSQPFDYWGQGTTLTVSS

SEQ ID NO: 32 - protein, 107 aa, Mus musculus
DIKMTQSPSSMYASLGERVTITCKASRDINTYLSWFQQKPGKSPKTLISRANRLVDGVPSRFSGSGSGQDYSLSISSLEYEDMGFYYCLQYDEFPWTFGGGTKLVIK

SEQ ID NO: 33 - protein, 117 aa, Mus musculus
QVQLKESGPGLVAPSQSLSITCTVSGFSLTRYGVHWVRQPPGKGLEWLGVIWAGGSTNYKSALMSRLNITKDNSKSQVFLKMNGLQTDDTAIYFCARAGGSSSFDYWGQGTTLTVSS

SEQ ID NO: 34 - protein, 107 aa, Mus musculus
DIKMTQSPSSMNASLGERVTITCKASQDINTYLGWFQQKPGKSPKTLIYRANRLVDGVPSRFSGSGSGQGYSLTISSLEYEDMGIYYCLQYDEFPWTFGGGTKLEIK

SEQ ID NO: 35 - protein, 117 aa, Mus musculus
QVQLKESGPGLVAPSQSLSITCTVSGFSLTGYGIHWVRQPPGKGLEWLGVIWADGTTNSNSALMSRLSISKDNSKSQVFLKMTSLQTDDTAMYHCARAGGSQPFDYWGQGTTLTVSS

SEQ ID NO: 36 - protein, 107 aa, Mus musculus
DIKMTQSPSSMYASLGERVTITCKASQDINSYLSWFQQKPGKSPKTLIFRANRLVDGVPSRFSGSGSGQDYSLTISSLEYEDMEIYYCLQYDEFPWTFGGGTKLEIK

SEQ ID NO: 37 - protein, 488 aa, Unknown
  Humanized Chimera
MCPMSSPQTLNTLTPTMGWSWIFLFLLSGTAGVRSEVQLQQSGPELVKPGTSMKISCKASGYSFTGYTMHWVKQSHGKNLEWIGLINPYNGGTGYNQKFKGKATLTVDKSSSTTYIELLSLTSEDSAVYYCARTSGYLFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 38 - protein, 241 aa, Unknown
  Humanized Chimera
MSGHSRNMKFPSQLLLLLLFGIPGMICDIQMTQSSSSFSVSLGDRVTITCKASEDIYNRLAWYQQKPGNAPRLLISGATSLETGVPSRFSGSGSGKDYIFSITSLQTEDVATYYCQQYWSSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPILKSFNRNEC

SEQ ID NO: 39 - protein, 447 aa, Unknown
  Humanized
QIHLVESGGGVVQTGKSMKISCKASGYSFTGYTMHWVKQTPGKNLEWIGLINPYNGGTGYNQKFKGKATLTVDKSSSTTYIELLSLTSEDSAVYYCARTSGYLFAYWGQGTLVTVSSASTKGPSVFPLAPSSKSTSGGTAALGCLVRDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVVHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Unknown
  Humanized
ETVMTQSPATLSVSPGERATITCKASEDIYNRLAWYQQKPGQAPRLLISGATSLETGVPSRFSGSGSGTEFTLTISSLQSEDVATYYCQQYWSSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 447 aa, Unknown
  Humanized
QVQLVQSGGQVKKPGASVKISCKASGYSFTGYTMHWVKQAPGRGLEWIGLINPYNGGTGYNQKFKGKATLTVDKSSSTTYIELLSLTSEDSAVYYCARTSGYLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 42 - protein, 214 aa, Unknown
  Humanized
DIVLTQTPDSLAVSLGERATITCKASEDIYNRLAWYQQKAGQPPKLLISGATSLETGVPDRFSGSGSGTDFTLTISSLQAEDVATYYCQQYWSSPLTFGAGTKVNIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 460 aa, Mus musculus
MAVLVLFLCLVAFPSCVLSQVQLRESGPGLVAPSQSLSITCTVSGFSLPRYGIHWVRQPPGKGLEWLGVIWAGGSTDYDSALMSRLSISKDTSKSQVFLKMSSLQTDDTAIYYCARAGGSQPFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 44 - protein, 236 aa, Mus musculus
MDMRTPAQFLGILLLWFPGIKCDIKMTQSPSSMYASLGERVTITCKASRDINTYLSWFQQKPGKSPKTLISRANRLVDGVPSRFSGSGSGQDYSLSISSLEYEDMGFYYCLQYDEFPWTFGGGTKLVIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEW

SEQ ID NO: 45 - protein, 460 aa, Mus musculus
MAVLVLFLCLVAFPSCVLSQVQLKESGPGLVAPSQSLSITCTVSGFSLTRYGVHWVRQPPGKGLEWLGVIWAGGSTNYKSALMSRLNITKDNSKSQVFLKMNGLQTDDTAIYFCARAGGSSSFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 46 - protein, 236 aa, Mus musculus
MDMRTPAQFLGILLLWFPGIKCDIKMTQSPSSMNASLGERVTITCKASQDINTYLGWFQQKPGKSPKTLIYRANRLVDGVPSRFSGSGSGQGYSLTISSLEYEDMGIYYCLQYDEFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 472 aa, Mus musculus
MAVLVLFLCLVAFPSCVLSQVQLKESGPGLVAPSQSLSITCTVSGFSLTGYGIHWVRQPPGKGLEWLGVIWADGTTNSNSALMSRLSISKDNSKSQVFLKMTSLQTDDTAMYHCARAGGSQPFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 48 - protein, 234 aa, Mus musculus
MRTPAQFLGILLLWFPGIKCDIKMTQSPSSMYASLGERVTITCKASQDINSYLSWFQQKPGKSPKTLIFRANRLVDGVPSRFSGSGSGQDYSLTISSLEYEDMEIYYCLQYDEFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEW

SEQ ID NO: 49 - DNA, 23 bp, Artificial Sequence
  Forward primer for A10 ab
aagcagtggtatcaacgcagagt

SEQ ID NO: 50 - DNA, 30 bp, Artificial Sequence
  Reverse primer for A10 ab and 9A7 ab
taggacctgagagctttgtgggtgctgagc

SEQ ID NO: 51 - DNA, 32 bp, Artificial Sequence
  Forward primer for A10 ab
ggggcggccgcagagtggccattacggccggg

SEQ ID NO: 52 - DNA, 35 bp, Artificial Sequence
  Reverse primer for A10 ab and 9A7 ab
ggggaattctcatttacccggagaccgggagatgg

SEQ ID NO: 53 - DNA, 20 bp, Artificial Sequence
  Forward primer for A10 ab CH
gcctccaccaagggcccatc

SEQ ID NO: 54 - DNA, 20 bp, Artificial Sequence
  Reverse primer for A10 ab CH
gcggccgcggtcaccaattc

SEQ ID NO: 55 - DNA, 36 bp, Artificial Sequence
  Forward primer for A10 ab VH
gaattggtgaccgcggccgcagagtggccattacgg

SEQ ID NO: 56 - DNA, 36 bp, Artificial Sequence
  Reverse primer for A10 ab VH
gggcccttggtggaggctgcagagacagtgaccaga

SEQ ID NO: 57 - DNA, 20 bp, Artificial Sequence
  Forward primer for A10 ab CL
cgaactgtggctgcaccatc

SEQ ID NO: 58 - DNA, 20 bp, Artificial Sequence
  Reverse primer for A10 ab CL
gcggccgcggtcaccaattc

SEQ ID NO: 59 - DNA, 36 bp, Artificial Sequence; identical to SEQ ID NO: 55
  Forward primer for A10 ab VL

SEQ ID NO: 60 - DNA, 41 bp, Artificial Sequence
  Reverse primer for A10 ab VL
gatggtgcagccacagttcgtttcagctccagcttggtccc

SEQ ID NO: 61 - DNA, 23 bp, Artificial Sequence
  Forward primer for  1G2 ab, 12D10 ab, 9A7 ab
aagcagtggtatcaacgcagagt

SEQ ID NO: 62 - DNA, 30 bp, Artificial Sequence
  Reverse primer for  1G2 ab, 12D10 ab
taggaccagagggctccaaggacactggga

SEQ ID NO: 63 - DNA, 32 bp, Artificial Sequence; identical to SEQ ID NO: 51
  Forward primer for  1G2 ab, 12D10 ab, 9A7 ab

SEQ ID NO: 64 - DNA, 35 bp, Artificial Sequence
  Reverse primer for  1G2 ab, 12D10 ab
ggggaattctcatttaccaggagagtgggagaggc